SEQUENCE LISTING (23 sequences)

SEQ ID NO: 1 - RNA, 19 nt, Artificial Sequence
  Synthetic oligonucleotide
agccgaaacucuucaucau

SEQ ID NO: 2 - RNA, 19 nt, Artificial Sequence
  Synthetic oligonucleotide
gaugcaggaucugcuuaga

SEQ ID NO: 3 - RNA, 21 nt, Artificial Sequence
  Synthetic oligonucleotide
aagcacagagagaagaaugag

SEQ ID NO: 4 - RNA, 21 nt, Artificial Sequence
  Synthetic oligonucleotide
aagaagcaggagaccaucgag

SEQ ID NO: 5 - RNA, 19 nt, Artificial Sequence
  Synthetic oligonucleotide
ggcucugaguaagaccuuu

SEQ ID NO: 6 - RNA, 21 nt, Artificial Sequence
  Synthetic oligonucleotide
aaggcacccuggcuucacucu

SEQ ID NO: 7 - RNA, 22 nt, Artificial Sequence
  Synthetic oligonucleotide
gaccugcagucccuccuucuuu

SEQ ID NO: 8 - RNA, 19 nt, Artificial Sequence
  Synthetic oligonucleotide
cuggacagguuguagauau

SEQ ID NO: 9 - protein, 16 aa, Artificial Sequence
  Synthetic polypeptide
RQIKIWFQNRRMKWKK

SEQ ID NO: 10 - protein, 27 aa, Artificial Sequence
  Synthetic polypeptide
GWTLNSAGYLLGKINLKALAALAKKIL

SEQ ID NO: 11 - protein, 16 aa, Artificial Sequence
  Synthetic polypeptide
PVIRVWFQNKRCKDKK

SEQ ID NO: 12 - protein, 13 aa, Artificial Sequence
  Synthetic polypeptide
GRKKRRQRRRPPQ

SEQ ID NO: 13 - protein, 16 aa, Artificial Sequence
  Synthetic polypeptide
LLIILRRRIRKQAHAH

SEQ ID NO: 14 - protein, 27 aa, Artificial Sequence
  Synthetic polypeptide
GALFLGWLGAAGSTMGAWSQPKKKRKV

SEQ ID NO: 15 - protein, 18 aa, Artificial Sequence
  Synthetic polypeptide
KLALKLALKALKAALKLA

SEQ ID NO: 16 - RNA, 19 nt, Artificial Sequence
  Synthetic polypeptide
gaaggcccauauagagaaa

SEQ ID NO: 17 - RNA, 19 nt, Artificial Sequence
  Synthetic oligonucleotide
gccaugcacuccugaguuu

SEQ ID NO: 18 - RNA, 20 nt, Artificial Sequence
  Synthetic oligonucleotide
ggguauuacucucaccgaga

SEQ ID NO: 19 - RNA, 20 nt, Artificial Sequence
  Synthetic oligonucleotide
gcuaugccaugaguacagaa

SEQ ID NO: 20 - RNA, 19 nt, Artificial Sequence
  Synthetic oligonucleotide
cacgccaagugguuuccaa

SEQ ID NO: 21 - RNA, 19 nt, Artificial Sequence
  Synthetic oligonucleotide
ccugggugaugcagaaacu

SEQ ID NO: 22 - RNA, 19 nt, Artificial Sequence
  Synthetic oligonucleotide
ccacauucaagaaaucaag

SEQ ID NO: 23 - RNA, 19 nt, Artificial Sequence
  Synthetic oligonucleotide
ccucgugaaugcacucauu

(19) United States
(12) Patent Application Publication
(10) Pub. No.: US 2009/0280058 A1
(43) Pub. Date: Nov. 12, 2009

(54) Delivery Of Double-Stranded RNA Into The Central Nervous System

(76) Inventors: Carol M. Troy, Hastings-on-Hudson, NY (US); Sander E. Connolly, New York, NY (US); Giselle F. Prunell, Montevideo (UY); Andrew F. Ducruet, New York, NY (US)

Correspondence Address: BAKER BOTTS L.L.P., 30 ROCKEFELLER PLAZA, 44TH FLOOR, NEW YORK, NY, 10112-4498, US

(21) Appl. No.: 12/397,478

(22) Filed: Mar. 4, 2009

Related U.S. Application Data
(63) Continuation of application No. PCT/US07/19605, filed on Sep. 7, 2007.
(60) Provisional application No. 60/845,048, filed on Sep. 15, 2006.

(51) Int. Cl. A61K 51/00; A61K 38/00; A61K 38/54

(52) U.S. Cl. 424/1.69; 514/2; 424/94.3

(57) ABSTRACT

The present invention provides for compositions and methods for in vivo delivery of a cell-permeable complex to cells of the central nervous system, wherein the cell-permeable complex decreases the level of a functional target protein encoded by a target mRNA molecule. In preferred embodiments of the invention, the cell-permeable complex comprises an siRNA nucleic acid molecule operably linked to a cell-penetrating peptide, wherein the cell-penetrating peptide facilitates transport of the cell-permeable complex across both the blood brain barrier and cell membrane of a target cell. The methods of the invention further encompass the utilization of convection-enhanced delivery methods such as intracerebral clysis (ICC) to deliver the cell-permeable complex to the target cells of the central nervous system.

### CROSS-REFERENCE TO RELATED APPLICATIONS

This application is a continuation of International Application PCT/U.S.07/019,605, filed Sep. 7, 2007, which claims priority to U.S. Provisional Application No. 60/845,048, filed Sep. 15, 2006, the entire contents of which are incorporated by reference herein.

### GRANT INFORMATION

This invention was made with government support under grant number R01 NS35933 awarded by the Institute of Neurological Disorders and Stroke. The government has certain rights in the invention.

### SEQUENCE LISTING

The specification further incorporates by reference the Sequence Listing submitted herewith. Pursuant to 37 C.F.R. § 1.52(e)(5), the Sequence Listing text file, identified as 0700503749SEQLIST.TXT, is 5,290 bytes and was created on Mar. 3, 2009.

### 1. INTRODUCTION

The present invention provides for compositions and methods for in vivo delivery of a cell-permeable complex to cells of the central nervous system, wherein the cell-permeable complex decreases the level of a functional target protein encoded by a target mRNA molecule. In preferred embodiments of the invention, the cell-permeable complex comprises an siRNA nucleic acid molecule operably linked to a cell-penetrating peptide, wherein the cell-penetrating peptide facilitates transport of the cell-permeable complex across both the blood brain barrier and cell membrane of a target cell. The methods of the invention further encompass the utilization of convection-enhanced delivery methods such as intracerebral clysis (ICC) to deliver the cell-permeable complex to the target cells of the central nervous system.

### 2. BACKGROUND OF THE INVENTION

### 2.1 Convection-Enhanced Drug Delivery

The blood-brain barrier prevents the delivery of many systemically-administered molecules to the brain (Chen et al., 2004, Curr Drug Deliv 1:361-376.). Current methods to improve drug delivery to the brain, including high-dose systemic injection, blood brain barrier modification, intra-arterial infusion, direct injection, infusion through an implanted reservoir, biodegradeable polymers, and intracerebroventricular infusion, have had some success but ultimately have been clinically inadequate (Jain, 1997, Adv Drug Deliv Rev 26:71-90). An inherent limitation of these delivery methods is reliance on diffusion to distribute the compound throughout the tissue. Drug distribution by diffusion occurs along a concentration gradient and is highly dependent on molecular weight. This process is slow and inefficient in brain tissue, particularly for charged, high molecular weight compounds (Jain, 1994, Sci Am 271:58-65; Jain, 1997, Adv Drug Deliv Rev 26:71-90). The lengthy time requirements for drug dispersion allow drug catabolism and tissue clearance to affect bioavailability. Additionally, the high source concentrations required to produce a concentration gradient can be toxic to the surrounding parenchyma (Kimler et al., 1992, J Neurooncol 14:191-200).

A convection-enhanced regional drug delivery method was developed which utilizes a positive-pressure, microinfusion mechanism to produce convective forces that distribute a therapeutic agent throughout the brain (Bobo et al., 1994, Proc Natl Acad Sci USA 91:2076-2080; Morrison et al., 1994, Am J Physiol 266:R292-305; Lieberman et al., 1995, J Neurosurg 82:1021-1029; Broaddus et al., 1998, J Neurosurg 88:734-742; Chen et al., 1999, J Neurosurg 90:315-320; Zirzow et al., 1999, Neurochem Res 24:301-305). This method, also referred to as “intracerebral clysis” (ICC), bypasses the blood-brain-barrier by establishing a positive pressure gradient in the brain via an implanted catheter attached to a microinfusion pump (Bruce et al., 2000, J Neurosurg 88:734-742). Bulk flow along the pressure gradient distributes the drug through the interstitial space and can be controlled by altering the infusion volume and/or rate (Chen et al., 1999, J Neurosurg 90:315-320; Bruce et al., 2000, J Neurosurg 88:734-742). Mathematical and experimental models have demonstrated the therapeutic advantages of ICC over commonly used diffusion-dependent methods such as systemic delivery and locally-placed biodegradable polymers (Jain, 1989, J Natl Cancer Inst 81:570-576). These advantages include greater volume of distribution, more uniform drug concentrations within the treatment volume, and relative independence from size and charge characteristics of the drug molecule. Additionally, the maximal volume of distribution achieved is attainable in a fraction of the time required for diffusion (Morrison et al., 1994, Am J Physiol 266:R292-305).

The focus of recent experimental and clinical applications of ICC has been in the delivery of therapeutic agents for brain tumors. Model studies in rodents have shown promising results for the delivery of minimally blood-brain barrier-permeable chemotherapeutic DNA synthesis inhibitors such as topotecan, gemcitabine, carboplatin, and temozolamide (Kaiser et al., 2000, Neurosurgery 47:1391-1399; Degen et al., 2003, J Neurosurg 99:893-898; Saito et al., 2004, Cancer Res 64:6858-6862). Further preclinical work has utilized ICC to deliver immunotoxin conjugates (Kawakami et al., 2004, J Neurosurg 101:1004-1011), boronated agents (Barth et al., 2004, Appl Radiat Isot 61:899-903; Yang et al., 2004, Appl Radiat Isot 61:981-985), as well as gene therapy in the treatment of tumors (Cunningham et al., 2000, Cell Transplant 9:585-594; Ohlfest et al., 2005, Mol Ther 12:778-788). The translational potential of ICC as a technique for delivering agents to the brain is evidenced by its use in a number of human trials. The first clinical trial of ICC for malignant glioma demonstrated the capacity of ICC to maximize therapeutic effect while limiting toxicity (Laske et al., 1997, Nat Med 3:1362-1368). Subsequent phase I/II clinical studies employing immunotoxin conjugates have also shown tumor specificity and adequate agent distribution with adverse effects similarly limited to target tissue damage and minimal toxicity (Rand et al., 2000, Clin Cancer Res 6:2157-2165; Kunwar, 2003, Acta Neurochir Suppl 88:105-111; Sampson et al., 2003, J Neurooncol 65:27-35; Weber et al., 2003, Acta Neurochir Suppl 88:93-103). Recent studies of paclitaxel via ICC utilized advanced imaging techniques to monitor convection of the drug in real time (Lidar et al., 2004, J Neurosurg 100:472-479; Popperl et al., 2005, Eur J Nucl Med Mol Imaging 32:1018-1025). ICC has also been used in a recent phase I/II trial of HSV-1-tk gene-containing liposomes (Voges et al., 2003, Ann Neurol 54:479-487). A recent trial reported in the literature is a Phase I/II trial of a chimeric monoclonal antibody to histone H (Patel et al., 2005, Neurosurgery 56:1243-1252). Clearly, ICC has potential for intraparenchymal delivery of various compounds including viruses, plasmids, antibodies, peptides, and oligonucleotides (Broaddus et al., 1998, J Neurosurg 88:734-742; Chen et al., 2005, J Neurosurg 103:311-319).

### 2.2 Transport Peptides

Peptide vectors have been used to deliver various macromolecules across plasma membranes. For example, published U.S. Pat. application 2002/0009758 discloses a means for transporting antisense nucleotides into cells using a short peptide vector, MPG. The MPG peptide contains a hydrophobic domain derived from the fusion sequence of HIV gp41, and a hydrophilic domain derived from the nuclear localization sequence of SV40 T-antigen. It has been demonstrated that several molecules of the MPG peptide coat the antisense oligonucleotide, which can then be delivered into cultured mammalian cells in less than 1 hour with relatively high efficiency (90%). Furthermore, it has been shown that the interaction with MPG strongly increases both the oligonucleotide's stability to nucleases, and its ability to cross the plasma membrane.

Penetratin-1 is a 16-amino-acid polypeptide derived from the third alpha-helix of the homeodomain of Drosophila antennapedia. Its structure and function have been well studied and characterized (see, e.g., Derossi, et al., 1998, Trends Cell Biol., 8(2), 84-87; Dunican, et al., 2001, Biopolymers, 60(1), 45-60; Hallbrink, et al., 2001, Biochim. Biophys. Acta, 1515(2), 101-109; Bolton, et al., 2000, Eur. J. Neurosci., 12(8), 2847-2855; Kilk, et al., 2001, Bioconjug. Chem., 12(6), 911-916); Bellet-Amalric, et al., 2000, Biochim. Biophys. Acta, 1467(1), 131-143; Fischer, et al., 2000, J. Pept. Res., 55(2), 163-172; Thoren, et al., 2000, FEBS Lett., 482(3), 265-268). It has been shown that Penetratin-1 efficiently carries avidin, a 63-kDa protein, into human Bowes melanoma cells (Kilk, et al., supra). Additionally, it has been shown that the transportation of Penetratin-1 and its cargo is non-endocytotic and energy-independent, and does not depend upon receptor molecules or transporter molecules. Furthermore, it is known that Penetratin-1 is able to cross a pure lipid bilayer (Thoren, et al., supra). This feature enables Penetratin-1 to transport its cargo, free from the limitation of cell-surface receptor/transporter availability. The delivery vector has been shown previously to enter all cell types (Derossi, et al., supra), and effectively deliver peptides (Troy, et al., 1996, Proc. Natl. Acad. Sci. USA, 93, 5635-5640) or antisense oligonucleotides (Troy, et al., 1996, J. Neurosci., 16, 253-261; Troy, et al., 1997, J. Neurosci., 17, 1911-1918).

Other cell-penetrating peptides that facilitate cellular uptake of attached molecules include transportan, pIS1, Tat(48-60), pVEC, MAP and MTS. Transportan is a 27 amino acid long peptide containing 12 functional amino acids from the amino terminus of the neuropeptide galanin and mastoparan in the carboxyl terminus, connected by a lysine (Pooga, et al., 1998, FASEB J., 12(1), 67-77). pIs1 is derived from the third helix of the homeodomain of the rat insulin 1 gene enhancer protein (Magzoub, et al., 2001, Biochim. Biophys. Acta, 1512(1), 77-89; Kilk, et al., 2001, Bioconjug. Chem., 12(6), 911-916).

Tat is a transcription activating factor of 86-102 amino acids that allows translocation across the plasma membrane of an HIV infected cell to transactivate the viral genome (Hallbrink, M., et al., 2001, Biochim Biophys Acta, 1515(2), 101-109; Suzuki, T., et al., 2002, J. Biol. Chem., 277(4), 2437-2443; Futaki, S., et al., 2001, J. Biol. Chem., 276(8), 5836-5840). A small Tat fragment extending from residues 48-60 has been determined to be responsible for nuclear import (Vives, et al., 1997, J. Biol. Chem., 272(25), 16010-16017). pVEC is an 18 amino acid long peptide derived from the murine sequence of the cell adhesion molecule vascular endothelial cadherin, extending from amino acid 615-632 (Elmquist et al., 2001, Exp. Cell Res., 269(2), 237-244). MTS, or membrane translocating sequences, are those portions of certain peptides which are recognized by acceptor proteins responsible for directing nascent translation products into the appropriate cellular organelles for further processing (Lindgren et al., 2000, Trends in Pharmacological Sciences, 21(3), 99-103; Brodsky, J. L., 1998, Int. Rev. Cyt., 178, 277-328; Zhao Y, et al., 2001, J. Immunol. Methods, 254(1-2), 137-145). An MTS of particular relevance is MPS peptide, a chimera of the hydrophobic terminal domain of the viral gp41 protein and the nuclear localization signal from simian virus 40 large antigen, which is one combination of nuclear localization signals and membrane translocation sequences that has been shown to internalize independent of temperature, and function as a carrier for oligonucleotides (Lindgren et al., 2000, Trends in Pharmacological Sciences, 21(3), 99-103; Morris et al., 1997, Nucleic Acids Res., 25, 2730-2736).

MAPs, or model amphipathic peptides, are a group of peptides having as their essential feature helical amphipathicity and a length of at least four complete helical turns. (Scheller, et al., 1999, J. Peptide Science, 5(4), 185-194; Hallbrink et al., 2001, Biochim Biophys Acta, 1515(2), 101-109).

U.S. Pat. No. 6,287,792 by Pardridge et al., discloses a method for delivering antisense oligonucleotides to cells by first linking the oligonucleotides to biotin. The biotinylated antisense oligonucleotides then bind to avidin/avidin fusion protein, which acts as a transportation vector to assist the antisense oligonucleotides in crossing cell membranes. U.S. Pat. No. 6,025,140 by Langel et al., discloses the use of vector peptides to deliver antisense molecules across plasma membranes, and specifically discloses the use of penetratin and transportan to transport peptide nucleic acids across cell membranes. Accordingly, the so called “cell-penetrating peptides” offer certain advantages for protocols involving the translocation of macromolecules into cells, including non-traumatic internalization, limited endosomal degradation, high translocation efficiencies at low concentrations, and delivery to a wide variety of cell types.

Published U.S. Pat. Application Nos. US2005/0260756 and US20060178297 both by Troy et al., have shown that a complex comprising the transport peptide Penetratin-1 covalently bound to siRNA can cross cell membranes and enter into cells in vitro. The complex exhibited a greater efficiency of transport across cell membranes as compared to conventional methods known in the art, such as transfection, electroporation, liposomal delivery, or microinjection. By facilitating the transport of siRNA into cells, the complex disclosed in US2005/0260756 provides a mechanism for temporarily decreasing the expression of genes targeted by the siRNA of the complex. This has advantages over transfection, or virus based delivery systems, which may have long-lasting effects since they may introduce a nucleic acid into a cell that is incorporated into the target cell's genome. Such mechanisms may be undesirable when only a temporary effect is desired.

Additionally, US2005/0260756 and US20060178297 describe the use of a complex comprising a transport peptide and an siRNA in genetic analysis. Engineering the siRNA portion of the complex to target a particular mRNA in a cell allows for the selective inhibition of the target mRNA's expression. Therefore, analyzing the phenotype of a cell in which a particular mRNA is selectively inhibited may reveal the normal cellular function of the inhibited mRNA, and its corresponding gene.

The peptide Penetratin1 has been used to deliver cargoes to the brain. The first report documenting the use of Penetratin-1 in the CNS showed that repeated intrathecal injections of an antisense oligonucleotide against the galanin receptor linked to Penetratin-1 resulted in a decrease in galanin binding in the dorsal horn and a functional suppression of galanin receptors (Pooga et al., 1998, Nat Biotechnol 16:857-861). Mode of delivery appears to be important in determining whether Penetratin-1 reaches the brain. Although the peptide did not reach the cerebral tissue when injected into rodents via the tail vein or into the cerebral ventricles (Bolton et al., 2000, Eur J Neurosci 12:2847-2855; Rousselle et al., 2000, Mol Pharmacol 57:679-686), its spread was dose dependant when injected into the striatum, with administration of 10 μg of the peptide resulting in a volume of spread of 1.61 mm³ (Bolton et al., 2000, Eur J Neurosci 12:2847-2855). Similarly, a 6-fold increase in the brain uptake of the anti-neoplastic agent doxorubicin was observed when it was coupled to Pen1 and injected into the carotid arteries of rats, without compromising the BBB integrity (Rousselle et al., 2000, Mol Pharmacol 57:679-686), suggesting that Pen1 is able to cross the BBB.

### 2.3 RNA Interference

RNA interference is an endogenous cellular mechanism that not only represses viruses, transposable elements, and repetitive genes, but also down-regulates genes post-transcriptionally in a very specific and efficient way (Ambros, 2004, Nature 431:350-355; Bender, 2004, Curr Opin Plant Biol 7:521-526; Ding et al., 2004, Virus Res 102:109-115; Lippman and Martienssen, 2004, Nature 431:364-370; Schramke and Allshire, 2004, Curr Opin Genet Dev 14:174-180). Researchers have taken advantage of its endogenous machinery to reduce the expression of molecules in different biological systems by exogenous administration of small interfering RNA (siRNA). Compared to the traditional approach of genetically modified animals to study gene function, it presents many advantages, including lack of compensations by other genes. The technique holds great promise for understanding the role of specific molecules in the normal and pathological brain, as well as a potential therapeutic tool to treat neurological diseases (Thakker et al., 2004, Proc Natl Acad Sci USA 101:17270-17275; Thakker et al., 2005b, Mol Psychiatry 10:782-789, 714; Wang et al., 2005, Neurosci Res 53:241-249).

The fairly recent discovery that RNA interference (RNAi) exists in mammals has opened the potential of using this mechanism for studying the function of individual gene products and also of applying RNAi to therapeutic uses. While an increasing number of studies have used RNAi in vivo, relatively few have employed RNAi in the mammalian brain. The successful delivery of siRNA to the neurons of the cerebral tissue is the first challenge for developing its potential as a therapeutic tool. Approaches to date have used local injection, transfection, electroporation, osmotic pumps, and viral delivery. Some studies have used synthetic siRNA and others have used vectors expressing short hairpin RNA (shRNA). However, none of these has proved optimal. So far, the different methods employed to deliver siRNA to cerebral tissue suffer many drawbacks (for a review see Thakker et al., 2005a, Pharmacol Ther. 109(3):413-38).

Naked siRNA does not cross the blood brain barrier (BBB) and has poor uptake by cells. When locally injected into the cerebral tissue distribution was restricted to a few cells in close proximity to the injection site (Makimura et al., 2002, BMC Neurosci 3:18; Shishkina et al., 2004, Neuroscience 129:521-528). An effective knockdown of genes in the adult mouse brain was reported when high siRNA amounts were injected into the cerebral ventricles over long periods of time (Thakker et al., 2004, Proc Natl Acad Sci USA 101:17270-17275; Thakker et al., 2005b, Mol Psychiatry 10:782-789, 714). Wider distribution and more efficient cellular uptake may be obtained with viral methods, lipid based transfection and injections of siRNA into the parenchyma followed by electroporation, but these techniques are associated with risk of oncogenesis or toxicity (Li et al., 2002, Science 296:497; Woods et al., 2003, Blood 101:1284-1289; Davidson et al., 2004, J Neurosci 24:10040-10046; Akaneya et al., 2005, J Neurophysiol 93:594-602; Hassani et al., 2005, J Gene Med 7:198-207; Wang et al., 2005, Neurosci Res 53:241-249).

Neurons have historically proven refractory to easy genetic manipulation. They are more resistant to transfection than most other cell types and, since they are post-mitotic, stable mutant cell lines cannot be established to counter these low efficiencies. Current techniques of transfection, such as lipid transfection reagents, induce substantial morbidity and mortality in primary hippocampal neuronal cultures (Davidson et al., 2004, J Neurosci 24:10040-10046) Published U.S. Pat. Application No. US2005/0234000 by Mitchell et al., discloses that siRNA trageting BDNF mRNA can reduce BDNF expression when microinjected into muscle tissue enervated by BDNF motor neurons in a cationic lipid delivery reagent. Similarly, U.S. Pat. No. 5,994,320 by Low et al., shows that siRNA directed to c-myb, epidermal growth factor (EGF), and the Platelet Derived Growth Factor (PDGF) inhibited tumor cell proliferation in the central nervous system when administered intratumorly via delivery agents such as liposomes. Additionally, viral infection using lentiviral or adenoviral vectors provides higher efficiencies than transfection but has the problems discussed above for in vivo applications. Finally, there may also be situations where it is not desirable to permanently alter expression of the targeted gene.

### 3. SUMMARY OF THE INVENTION

The present invention provides for compositions and methods that utilize a cell-permeable complex for facilitating the delivery of a double-stranded ribonucleic acid molecule into a central nervous system cell to reduce the expression of a target protein. Specifically, the invention provides a cell-permeable complex that comprises a double-stranded ribonucleic acid molecule effective in inhibiting the expression of a target protein encoded by a target mRNA expressed in the central nervous system, operably linked to a cell-penetrating peptide. The present invention also provides for methods of delivering the cell-permeable complex to target cells of the central nervous system, such as convection-enhanced delivery systems. In one non-limiting embodiment, the convection-enhanced delivery system is intracerebral clysis (ICC).

The methods of the present invention comprise, in non-limiting embodiments, introducing a cell-permeable complex to the central nervous system by a convection enhanced delivery method such as ICC, wherein the cell-permeable complex comprises an siRNA directed to a target mRNA so as to decrease the level of the target mRNA and its encoded target protein.

The present invention further provides methods of treating disorders and injuries of the central nervous system. For example, in one set of non-limiting embodiments, the methods of the invention may be used to promote apoptosis of tumor cells and/or decrease the growth of a tumor in the central nervous system. In specific non-limiting embodiments, the present invention may be used to decrease the expression of an apoptosis-inhibiting target protein such as, but not limited to, XIAP, cIAP1 and cIAP2. Accordingly, the invention, in specific non-limiting embodiments, may be used to increase the activity of pro-apoptotic proteins in a tumor cell, for example, but not limited to, Caspase-3, Caspase-7, Caspase-8, and Caspase-9.

In a further set of non-limiting embodiments, the present invention provides for methods of treating cerebral ischemia. In specific non-limiting embodiments, the invention may be used to inhibit neuronal death due to ischemia, for example, by inhibiting the expression of pro-apoptotic proteins, such as, but not limited to, caspase 2, caspase 3, caspase 6, caspase 7, caspase 8, caspase 9, PIDD, RAIDD, and NNOS.

### 4. BRIEF DESCRIPTION OF THE FIGURES

FIG. 1A-F. FITC-labeled siRNA to Caspase-8 linked to Penetratin-1 (V-siRNA-FITC) is rapidly taken up by cultured hippocampal neurons and distributed in the cytoplasm (FIG. 1A-C), while FITC-labeled siRNA not linked to Penetratin-1 was not taken up by the hippocampal neurons (FIG. 1D-F).

FIG. 2A-E. siRNA were directed to each of Caspase-1, Caspase-2, RAIDD, PIDD, and NNOS mRNA. Each of the Pen1-siRNA were linked to Penetratin-1. Cultured hippocampal neurons were treated with and without Pen1-siRNA. Western Blotting analysis shows that Pen1-siRNA reduced the expression of targeted mRNA as compared to untreated control cells (Co). Pen1-siRNA were targeted to the following mRNA's: Caspase-1 (siC1) (FIG. 2A); Caspase-2 (siC2) (FIG. 2B); RAIDD (FIG. 2C); PIDD (FIG. 2D); and NNOS (FIG. 2E). Arrows indicate the protein bands showing reduced expression.

FIG. 3A-D. Pen1-siRNA provides for a highly efficient, minimally toxic method of delivering siRNA to neurons. Pen1-siRNA is specific for targeted Caspase. Only the targeted Caspase (green) is down-regulated, not other family members (red). Caspase-8 expression is reduced by Pen1-siRNA directed to Caspase-8 mRNA (V-Casp8i) (FIG. 3A-B). Caspase-9 expression was reduced by Pen1-siRNA targeting Caspase-9 mRNA (V-Casp9i) (FIG. 3C-D).

FIG. 4A-B. Pen1-siRNA targeting Caspase-3 (Pen1-siCASP3) reduced the expression of Caspase-3 in primary rat hippocampal cultures. Caspase-3 is conserved in rats and mice. After one day treatment there was substantial down-regulation of the protein encoded by the targeted Caspase-3 mRNA (FIG. 4B) when compared to control cells not treated with Pen1-siCASP3 (FIG. 4A), as visualized by immunocytochemistry (green=Caspase-3, red=cytochrome c). A 90% reduction of the Caspase-3 mRNA after one day treatment was also detected by RealTime PCR.

FIG. 5A-B. Pen1-siRNA targeting XIAP open reading frame (ORF) (Pen1-siXIAP) reduced the expression of XIAP in primary rat hippocampal cultures. The ORF region targeted is conserved in rats and mice. After one day treatment there was substantial down-regulation of the protein encoded by the targeted XIAP mRNA (FIG. 5B) when compared to control cells not treated with Pen1-siXIAP (FIG. 5A), as visualized by immunocytochemistry (green=XIAP, red=cytochrome c). An 80% reduction of the XIAP mRNA after one day treatment was also detected by RealTime PCR.

FIG. 6A-E. Variation in ICP during ICC with four different flow rates. ICP was measure as a total volume of 100 μl of a 25% albumin solution was infused through ICC at rates of 0.5, 1.0, 2.0, 3.0, 4.0 μl/min. ICP was measured at the five flow rates in animals following implantation of tumor cells. Measurements were made at 0 days post tumor cell implantation (FIG. 6A), 10 days post tumor cell implantation (FIG. 6B), 15 days post tumor cell implantation (FIG. 6C), 20 days post tumor cell implantation (FIG. 6D) and 25 days post tumor cell implantation (FIG. 6E) post transplantation. ICP changes associated with rates of 0.5 and 1.0 μl/min were significantly smaller than those associated with flow rates of 2.0-4.0 μl/min.

FIG. 7A-B. The distribution of FITC-dextran after delivery via ICC at a flow rate of 3.0 μl showing macromolecule distribution patterns in the rat brain. FIG. 7A shows the distribution alter the infusion of a total of 10 μl. FIG. 7B shows the distribution after infusion with 30 μl.

FIG. 8. The cross-sectional areas of fluorescence in representative brain sections were compared for animals sacrificed at various time points, as indicated, following ICC. A total volume of 10 μl or 30 μl was administered through ICC. Each of the two volumes were administered at flow rates of 0.5 μl/min and 3.0 μl/min. A statistically significant difference in distribution between the 10 μl and 30 μl infusion groups, independent of infusion rate and post-infusion period, was observed, wherein the 30 μl volume exhibited a greater area of distribution than the 10 μl volume.

FIG. 9. Pen1-siRNA is delivered to the central nervous system with the clysis method. Rhodamine-labeled Pen1-siRNA was administered to the right side of the brain of an adult rat. There is substantial uptake of rhodamine-Pen1-siRNA on the right side of the brain while there is no detectable uptake on the left side. The rhodamine label is on the siRNA and is detected within cells and processes.

### 5. DETAILED DESCRIPTION OF THE INVENTION

For purposes of clarity, and not by way of limitation, the detailed description of the invention is divided into the following subsections:

(i) cell-permeable complexes;

(ii) delivery of the cell-permeable complex to cells;

(iii) conditions to be treated; and

(iv) examples.

### 5.1 Cell-Permeable Complexes

The present invention provides for a cell-permeable complex for facilitating the delivery of a double-stranded ribonucleic acid molecule into a cell, as well as various uses of the complex. Specifically, it has been found that a cell-penetrating peptide may be operably-linked to a double-stranded ribonucleic acid molecule to form a cell-permeable complex. Advantageously, the use of the complex yields an unprecedented and unexpected 100% transfection efficiency of dsRNA into neuronal cells. Such unprecedented uptake efficiency allows for the efficient in vivo delivery of dsRNA into tissues, and by extension, into entire organisms, thereby expanding the therapeutic possibilities of RNA interference applications. While the present invention is primarily directed to the delivery of a double-stranded ribonucleic acid molecule into a cell for the purposes of RNA interference, the cell-permeable complex described herein may also be used to facilitate the delivery of other non-coding RNAs, such as small temporal RNAs, small nuclear RNAs, small nucleolar RNAs or microRNAs, which may be used in applications other than RNA interference.

In specific, non-limiting embodiments, the present invention provides for a solution, suitable for instillation into the CNS, comprising a cell-permeable complex of the invention at a concentration of between 1 and 500 μM, more preferably between 10 and 200 μM, more preferably between 20 and f 100 μM, and most preferably 80 μM.

In specific, non-limiting embodiments, the present invention provides for a composition comprising an effective amount of a cell-permeable complex of the invention in a pharmaceutically acceptable solvent or solution (for example, sterile water or a solution comprising saline, a saline/glucose solution, etc.) further comprising albumin (e.g. human albumin, e.g., human serum albumin or HSA), for example comprising between 0.1 and 75% albumin, or between 1 and 75% albumin, or between 5 and 50% albumin, or between 10 and 30% albumin, and most preferably 25% albumin, where the percent is weight/volume. Such solution may comprise, for example but not by way of limitation, cell-permeable complex in a concentration of between 1 and 500 μM, more preferably between 10 and 200 μM, more preferably between 20 and 100 μM, and most preferably 80 μM.

The term “cell-permeable,” as used herein, means that, for a complex of the invention, the complex comprising transport peptide and dsRNA has substantially greater intracellular uptake than the dsRNA alone, e.g., uptake is increased by at least about 20, 30, 40 or 50 percent.

The following subsections described the RNA and peptide components of the cell-permeable complex. The RNA and peptide components are operably linked, meaning that they are joined, directly or indirectly, such that each is able to perform its desired function. Indirect joining utilizes a linker molecule, which may be a nucleic acid or nucleic acid derivative, an amino acid, peptide, amino acid derivative, or peptidomimetic, or other molecule with functionalities which permit joining the RNA and peptide components.

### 5.1.1 RNA Component of the Cell-Permeable Complex

In a non-limiting embodiment, the cell-permeable complex described herein comprises a double-stranded ribonucleic acid molecule operably linked to a cell-penetrating peptide.

In a further non-limiting embodiment, a “double-stranded ribonucleic acid molecule” refers to any RNA molecule comprising a double stranded portion, (e.g., containing an RNA duplex), notwithstanding the presence of single stranded gaps or overhangs of unpaired nucleotides. Further, as used herein, a double-stranded ribonucleic acid molecule includes single stranded RNA molecules forming functional stem-loop structures, such as small temporal RNAs, short hairpin RNAs and microRNAs, thereby forming the structural equivalent of an RNA duplex with single strand overhangs. The RNA molecule of the present invention may be isolated, purified, native or recombinant, and may be modified by the addition, deletion, substitution and/or alteration of one or more nucleotides, including non-naturally occurring nucleotides or deoxyribonucleotides, including those added at 5′ and/or 3′ ends to increase nuclease resistance.

The double-stranded ribonucleic acid molecule of the cell-permeable complex may be any one of a number of non-coding RNAs (i.e., RNA which is not mRNA, tRNA or rRNA), including, preferably, a small interfering RNA, but may also comprise a small temporal RNA, small nuclear RNA, small nucleolar RNA, short hairpin RNA or a microRNA comprising a double-stranded structure and/or a stem loop configuration comprising an RNA duplex with or without one or more single strand overhang. The double-stranded RNA molecule may be very large, comprising thousands of nucleotides, or preferably in the case of RNAi protocols involving mammalian cells, may be small, in the range of 21-25 nucleotides. In the dsRNA molecules of the invention, at least one strand comprises a portion homologous to the target gene, where said homologous portion is between about 5 and 50, 10 and 30, or 15 and 28 nucleotides in length. In a preferred embodiment, dsRNA of the present invention comprises a double-stranded RNA duplex of at least 19 nucleotides, and even more preferably, comprises a 21 nucleotide sense and a 21 nucleotide antisense strand paired so as to have a 19 nucleotide duplex region and a 2 nucleotide overhang at each of the 5′ and 3′ ends. Even more preferably, the 2 nucleotide 3′ overhang comprises 2′ deoxynucleotides, e.g., TT, for improved nuclease resistance.

As used herein, “homologous” refers to a nucleotide sequence that has at least 80% sequence identity, preferably at least 90%, at least 95%, or at least 98% sequence identity, or 100% sequence identity, to a portion of mRNA transcribed from the target gene. Homology may be determined using standard software such as BLAST or FASTA.

In a preferred non-limiting embodiment of the invention, a portion of at least one strand of the double-stranded ribonucleic acid molecule (i.e., the antisense strand) of the cell-permeable complex is homologous to a portion of mRNA transcribed from the Caspase-1 gene. In non-limiting examples, the Caspase-1 gene can be human (GenBank Accession Nos. NM_{—}033293, NM_{—}033295 NM_{—}033292, NM_{—}001223, NM_{—}033294, BC062327, and AK223503), mouse (GenBank Accession Nos. NM_{—}009807, and BC008152), or rat (GenBank Accession No. NM_{—}012762) Caspase-1. In one non-limiting embodiment, the double-stranded ribonucleic acid is a small interfering RNA targeted to the nucleotide sequence of (sequence of sense strand shown) of GAA GGC CCA UAU AGA GAA A (SEQ ID NO: 16) (GenBank accession number BC008152, initiation at base 201, target bases 1151-1169; GenBank accession number NM_{—}012762, initiation at base 1, target bases 951-969).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the Caspase-2 gene. In non-limiting examples, the Caspase-2 gene can be human (GenBank Accession Nos. NM_{—}032983, NM_{—}032982, BC002427, CR541748, AY889376, AY889375, AY888697, AY893402, BT007240, and AY219042), mouse (GenBank Accession Nos. NM_{—}007610, and BC034262), or rat (GenBank Accession No. NM_{—}022522) Caspase-2. In one non-limiting embodiment, the double-stranded ribonucleic acid is a small interfering RNA targeted to the nucleotide sequence of (sequence of sense strand shown) of GCC AUG CAC UCC UGA GUU U (SEQ ID NO: 17) (GenBank accession number NM_{—}007610, initiation at base 86, target bases 616-634; GenBank accession number NM_{—}022522, initiation at base 7, target bases 537-555).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the Caspase-3 gene. In non-limiting examples, the Caspase-3 gene can be human (GenBank Accession Nos. NM_{—}032991 and NM_{—}004346), mouse (GenBank Accession Nos. NM_{—}009810, BC038825, and Y13086), or rat (GenBank Accession Nos. NM_{—}012922 and NM_{—}022522) Caspase-3. In one non-limiting embodiment, the double-stranded ribonucleic acid is a small interfering RNA targeted to the nucleotide sequence of (sequence of sense strand shown) of AGC CGA AAC UCU UCA UCA U (SEQ ID NO: 1) (GenBank accession number BC038825, initiation at base 111, target bases 569-589; GenBank accession number NM_{—}012922, initiation at base 57, target bases 517-535).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the Caspase-6 gene. In non-limiting examples, the Caspase-6 gene can be human (GenBank Accession Nos. NM_{—}001226, NM_{—}032992, BC000305, BC004460, and AY254046), mouse (GenBank Accession Nos. BC002022 and NM_{—}009811), or rat (GenBank Accession Nos. BC078785, NM_{—}031775 and AF025670), Caspase-6. In one non-limiting embodiment, the double-stranded ribonucleic acid is a small interfering RNA targeted to the nucleotide sequence of (sequence of sense strand shown) of GGG UAU UAC UCU CAC CGA GA (SEQ ID NO: 18) (GenBank accession number BC002022, initiation at base 57, target bases 645-665; GenBank accession, number BC078785, initiation at base 187, target bases 778-797).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the Caspase-7 gene. In non-limiting examples, the Caspase-7 gene can be human (GenBank Accession Nos. NM_{—}033338, NM_{—}033339, NM_{—}001227, and NM_{—}033340) mouse (GenBank Accession Nos. BC005428 and NM_{—}007611), or rat (GenBank Accession Nos. BC070936 and NM_{—}022260), Caspase-7. In one non-limiting embodiment, the double-stranded ribonucleic acid is a small interfering RNA targeted to the nucleotide sequence (sequence of sense strand shown) of GAU GCA GGA UCU GCU UAG A (SEQ ID NO:2) (GenBank accession number BC070936, initiation at base 3, target bases 356-374).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the Caspase-8 gene. In non-limiting examples, the Caspase-8 gene can be human (GenBank Accession Nos. NM_{—}033358, NM_{—}033356, NM_{—}033355 NM_{—}001228, BC068050, BC028223, BC017031, and BC010390), mouse (GenBank Accession Nos. BC006737, NM_{—}009812, BC049955, and CT010166), or rat (GenBank Accession No. NM_{—}022277) Caspase-8. In further non-limiting embodiments, the double-stranded ribonucleic acid is a small interfering RNA targeted to the nucleotide sequences (sequence of sense strand shown) of AAG CAC AGA GAG AAG AAU GAG (SEQ ID NO:3) (GenBank Accession No. BC006737, initiation at base 336, target bases 878-898); AAG AAG CAG GAG ACC AUC GAG (SEQ ID NO:4) (GenBank Accession No. BC006737, initiation at base 336, target bases 432-452); or GGC UCU GAG UAA GAC CUU U, (SEQ ID NO:5) (GenBank accession number BC006737, initiation at base 336, target bases 1145-1163, GenBank accession number NM_{—}0222771, initiation at base 318, target bases 1127-1145).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the Caspase-9 gene. In non-limiting examples, the Caspase-9 gene can be human GenBank Accession Nos. NM_{—}032996, NM_{—}001229, BC002452, BC006463, AY732490, AY892274, AY889808, BT006911, AY214168, and AF093130), mouse (GenBank Accession Nos. NM_{—}015733, BC056447, BC056372, and CT010400) or rat (GenBank Accession No. NM_{—}031632), Caspase-9. In further non-limiting embodiments, the double-stranded ribonucleic acid is a small interfering RNA targeted to the nucleotide sequences (sequence of sense strand shown) of AAG GCA CCC UGG CUU CAC UCU (SEQ ID NO:6) (GenBank Accession No. NM015733, initiation at base 244, target bases 488-508); GAC CUG CAG UCC CUC CUU CUU U, (SEQ ID NO:7) GenBank Accession No. NM015733, initiation at base 244, target bases 1492-1511).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the XIAP gene. In non-limiting examples, the XIAP gene can be human (GenBank Accession Nos., U45880, and X99699), mouse (GenBank Accession No, U88990), or rat XIAP. In further non-limiting embodiments, the double-stranded ribonucleic acid is a small interfering RNA targeted to the nucleotide sequence (sequence of sense strand shown) of CUG GAC AGG UUG UAG AUA U (SEQ ID NO: 8) (GenBank Accession Number NM_{—}009688, initiation at base 672, target bases 1099-1117, GenBank Accession Number AB033366, initiation at base 330, target bases 757-775).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the cIAP1 (Inhibitor of Apoptosis) gene. In non-limiting examples, the cIAP1 gene can be human (GenBank Accession No. NM_{—}001166, BC016174, BC028578, and DQ068066), mouse (Genbank Accession No. NM_{—}007465), or rat (GenBank Accession No. BC062055, NM_{—}021752, and AF190020), cIAP1. In one non-limiting embodiment, the double-stranded ribonucleic acid is a small interfering RNA targeted to the nucleotide sequence of (sequence of sense strand shown) of GCU AUG CCA UGA GUA CAG AA (SEQ ID NO: 19) (GenBank accession number NM_{—}007465, initiation at base 779, target bases 1290-1309; GenBank accession number AF190020, initiation at base 1015, target bases 1463-1482).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the cIAP2 (Inhibitor of Apoptosis) gene. In non-limiting examples, the cIAP2 gene can be human (GenBank Accession No. NM_{—}182962, NM_{—}001165, BC037420, BC027485, and AY764389), mouse (GenBank Accession No. BC011338 and NM_{—}007464), or rat (GenBank Accession No. BC083555), cIAP2. In one non-limiting embodiment, the double-stranded ribonucleic acid is a small interfering RNA targeted to the nucleotide sequence of (sequence of sense strand shown) of CAC GCC AAG UGG UUU CCA A (SEQ ID NO:20) (GenBank accession number BC083555, initiation at base 233, target bases 1166-1184).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the PIDD (p 53 Induced Protein with Death Domain) gene. In non-limiting examples, the PIDD gene can be human (GenBank Accession No. AF274972), mouse (GenBak Accession No. AF274973), or rat PIDD. In one non-limiting embodiment, the double-stranded ribonucleic acid is a small interfering RNA targeted to the nucleotide sequence of (sequence of sense strand shown) of CCU GGG UGA UGC AGA AAC U (SEQ ID NO:21) (GenBank accession number AF274973, initiation at base 79, target bases 2427-2445).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the RAIDD ((RIP)-associated ICH-1/CED-3 Homologous Protein with a Death Domain) gene. In non-limiting examples, the RAIDD gene can be human (GenBank Accession No. NM_{—}003805, BC017042, and BT009837), mouse (GenBank Accession Nos. NM_{—}009950, MMAJ4740, MMAJ4738, BC005608, and NM_{—}009950), or rat (GenBank Accession Nos. XM_{—}001080418 and XM_{—}235061), RAIDD. In one non-limiting embodiment, the double-stranded ribonucleic acid is a small interfering RNA targeted to the nucleotide sequence of (sequence of sense strand shown) of CCA CAU UCA AGAAAU CAA G (SEQ ID NO:22) (GenBank accession number MMAJ4740, initiation at base 105, target bases 221-238; and GenBank accession number XM_{—}001080418, initiation at base 112, target bases 228-245).

In another preferred, non-limiting embodiment of the invention, a portion of at least one strand of the double-stranded ribonucleic acid molecule (i.e., the antisense strand) of the cell-permeable complex is homologous to a portion of mRNA transcribed from the NNOS (Neuronal Nitric Oxide Synthase) gene. In non-limiting examples, the NNOS gene can be human (GenBank Accession No. AK002203, NM_{—}014697, NM_{—}000620, BC014189, BC041382, BC112295, and AH005382), mouse (GenBak Accession No. NM_{—}008712), or rat (GenBank Accession No. X59949) NNOS. In one non-limiting embodiment, the double-stranded ribonucleic acid is a small interfering RNA targeted to the nucleotide sequence of (sequence of sense strand shown) of CCU CGU GAA UGC ACU CAU U (SEQ ID NO:23) (GenBank accession number NM_{—}008712, initiation at base 79, target bases 2427-2445; GenBank accession number: X59949, initiation at base 349, target bases 3474-3492.).

In the practice of the present invention, at least one strand of the double-stranded ribonucleic acid molecule (either the sense or the antisense strand) may be modified for linkage to a cell-penetrating peptide, for example, with a thiol group, so that a covalent bond may join the modified strand to the cell-penetrating peptide. Where the strand is modified with a thiol group, the covalent bond linking the cell-penetrating peptide and the modified strand of the ribonucleic acid molecule can be a disulfide bond, as is the case where the cell-penetrating peptide has a free thiol function (i.e., pyridyl disulfide or a free cysteine residue) for coupling. However, it will be apparent to those skilled in the art that a wide variety of functional groups may be used in the modification of the ribonucleic acid, so that a wide variety of covalent bonds may be applicable, including, but not limited to, ester bonds, carbamate bonds and sulfonate bonds.

In a preferred embodiment of the invention, it is the 5′ end of at least one strand of the double-stranded ribonucleic acid that is modified for linkage with the cell-penetrating peptide, for instance, with a group having a thiol function (e.g., a 5′ amino-C6 linker), thereby leaving the 3′ OH end of the strand free. Alternatively, where activity of the double-stranded ribonucleic acid molecule is not adversely affected (i.e., there is no significant reduction in degradation of target mRNA), at least one strand of the double-stranded ribonucleic acid may be modified at its 3′ end for linkage with the cell-penetrating peptide, where the covalent bond links the 3′ modified strand to the cell-penetrating peptide (Holen, T., et al., 2002, Nucleic Acids Res., 30(8), 1757-1766).

A label may also be affixed to at least one strand of the double-stranded ribonucleic acid molecule, including an enzyme label, a chemical label, or a radioactive label. Common enzymatic labels include horseradish peroxidase, biotin/avidin/streptavidin labeling, alkaline phosphatase and beta-galactosidase. Chemical labels include fluorescent agents, such as fluorescein and rhodamine, fluorescent proteins, such as phycocyanin or green fluorescent protein, and chemiluminescent labels. Fluorescein may be linked to the ribonucleic acid by using the reactive derivative fluorescein isothiocyanate (FITC). Finally, common radioactive labels include ³H, ¹³¹I and ⁹⁹Tc. In one specific, non-limiting embodiment, the label is affixed to the 5′ end of the strand, although the label may be attached at the 3′ end of the strand where such attachment does not significantly affect the activity of the double-stranded ribonucleic acid molecule.

In particular non-limiting embodiments, at least one strand of the double-stranded ribonucleic acid molecule is modified at its 5′ end for linkage with the cell-penetrating peptide, and a covalent bond links the 5′ modified strand to the cell-penetrating peptide. The 5′ end may be modified with a group having a thiol function, and the covalent bond linking the modified 5′ end with the cell-penetrating peptide may be a disulfide bond, such as would be the case where the cell-penetrating peptide has a free thiol group or group of corresponding function for attachment. Alternatively, where function of the double-stranded ribonucleic acid molecule is not adversely affected by such modification, at least one strand of the double-stranded ribonucleic acid molecule may be modified at its 3′ end for linkage with the cell-penetrating peptide, where the covalent bond links the 3′ modified strand to the cell-penetrating peptide.

### 5.1.2 Cell-Penetrating Peptide Component of the Cell-Permeable Complex

The cell-permeable complex described herein comprises a cell-penetrating peptide operably linked to a double-stranded ribonucleic acid molecule. Several features make cell-penetrating peptides unique vehicles for transporting biologically important molecules into cells. In particular, the activity of cell-penetrating peptides is generally non-cell-type specific. Additionally, cell-penetrating peptides typically function with high efficiency, even at low concentrations. Furthermore, the penetration of cell-penetrating peptides through cell membranes may be (but is not necessarily) independent of endocytosis, energy requirements, receptor molecules, and transporter molecules. Thus, cell-penetrating peptides can efficiently deliver large cargo molecules into a wide variety of target cells (Derossi, et al., 1998, Trends Cell Biol., 8(2), 84-87; Dunican, et al., 2001, Biopolymers, 60(1), 45-60; Hallbrink, et al., 2001, Biochim. Biophys. Acta, 1515(2), 101-109; Bolton, et al., 2000, Eur. J. Neurosci., 12(8), 2847-2855; Kilk, et al., 2001, Bioconjug. Chem., 12(6), 911-916).

As used herein, a “cell-penetrating peptide” is a peptide that comprises a short (about 8-50 or about 12-30 residues) amino acid sequence or functional motif that confers the energy-independent (i.e., non-endocytotic) translocation properties associated with the transport of the cell-permeable complex across the plasma and/or nuclear membranes of a cell. The cell-penetrating peptide used in the cell-permeable complex of the present invention preferably comprises at least one non-functional cysteine residue free or derivatized to form a disulfide link with a double-stranded ribonucleic acid which has been modified for such linkage. Representative amino acid motifs conferring such properties are listed in U.S. Pat. No. 6,348,185, the contents of which are incorporated herein by reference. The cell-penetrating peptides of the present invention preferably include, but are not limited to, Penetratin-1, transportan, pIs1, TAT (48-60), pVEC, MTS and MAP.

In the most preferred embodiment, the cell-penetrating peptide of the cell-permeable complex is Penetratin-1 (Pen1), comprising the peptide sequence RQIKIWFQNRRMKWKK (SEQ ID NO:9), conservative variants thereof, and peptides which are at least about 80%, or about 85%, or about 90%, or about 95%, homologous thereto (using standard homology-determining techniques such as BLAST or FASTA) and which retain an ability to promote cellular uptake of a linked cargo molecule. As used herein, a “conservative variant” is a peptide having one or more amino acid substitutions, wherein the substitutions do not adversely affect the shape, and therefore, the biological activity (i.e., transport activity) or membrane toxicity of the cell-penetrating peptide.

In specific non-limiting embodiments of the invention, the cell-permeable complex comprises Penetratin-1 operably linked to a double-stranded siRNA nucleic acid molecule homologous to mRNA encoding Caspase-1, Caspase-2, Caspase-3, Caspase-6, Caspase-7, Caspase-8, Caspase-9, XIAP, cIAP1, cIAP2, RAIDD or PIDD (Pen1-siCasp1, Pen1-siCasp2, Pen1-siCasp3, Pen1-siCasp6, Pen1-Casp7, Pen1-Casp8, Pen1-Casp9, Pen1-XIAP, Pen1-sicIAP1, Pen1-sicIAP2, Pen1-siRAIDD and Pen1-siPIDD, respectively).

The invention also provides for other cell-penetrating peptides that can be used including, but not limited to, transportan, pIS1, Tat(48-60), pVEC, MAP and MTS.

In one non-limiting embodiment, the cell-penetrating peptide is Transportan, wherein the peptide comprises the amino acid sequence GWTLNSAGYLLGKINLKALAALAKKIL (SEQ ID NO:10), conservative variants thereof, and peptides which are at least about 80%, or about 85%, or about 90%, or about 95%, homologous thereto (using standard homology-determining techniques such as BLAST or FASTA) and which retain an ability to promote cellular uptake of a linked cargo molecule.

In one non-limiting embodiment, the cell-penetrating peptide is pIs1, wherein the peptide comprises the amino acid sequence PVIRVWFQNKRCKDKK (SEQ ID NO:11), conservative variants thereof, and peptides which are at least about 80%, or about 85%, or about 90%, or about 95%, homologous thereto (using standard homology-determining techniques such as BLAST or FASTA) and which retain an ability to promote cellular uptake of a linked cargo molecule.

In one non-limiting embodiment, the cell-penetrating peptide is Tat, wherein the peptide comprises the amino acid sequence GRKKRRQRRRPPQ (SEQ ID NO:12), conservative variants thereof, and peptides which are at least about 80%, or about 85%, or about 90%, or about 95%, homologous thereto (using standard homology-determining techniques such as BLAST or FASTA) and which retain an ability to promote cellular uptake of a linked cargo molecule.

In one non-limiting embodiment, the cell-penetrating peptide is pVEC, wherein the peptide comprises the amino acid sequence LLIILRRRIRKQAHAH (SEQ ID NO: 13), conservative variants thereof, and peptides which are at least about 80%, or about 85%, or about 90%, or about 95%, homologous thereto (using standard homology-determining techniques such as BLAST or FASTA) and which retain an ability to promote cellular uptake of a linked cargo molecule.

In one non-limiting embodiment, the cell-penetrating peptide an MTS peptide, for example, but not limited to MPS, wherein the MPS peptide comprises the amino acid sequence GALFLGWLGAAGSTMGAWSQPKKKRKV (SEQ ID NO:14), conservative variants thereof, and peptides which are at least about 80%, or about 85%, or about 90%, or about 95%, homologous thereto (using standard homology-determining techniques such as BLAST or FASTA) and which retain an ability to promote cellular uptake of a linked cargo molecule.

In one non-limiting embodiment, the cell-penetrating peptide is MAPs (model amphipathic peptides), wherein the peptide comprises the amino acid sequence KLALKLALKALKAALKLA-amide (SEQ ID NO:15), conservative variants thereof, and peptides which are at least about 80%, or about 85%, or about 90%, or about 95%, homologous thereto (using standard homology-determining techniques such as BLAST or FASTA) and which retain an ability to promote cellular uptake of a linked cargo molecule.

The cell-penetrating peptides and the double-stranded ribonucleic acids described above are operably linked to form the cell-permeable complex of the present invention. The general strategy for conjugation is to prepare the cell-penetrating peptide and double-stranded ribonucleic acid components separately, each modified or derivatized with appropriate reactive groups to allow for linkage between the two. The modified double-stranded ribonucleic acid is then incubated together with a cell-penetrating peptide that is prepared for linkage, for a sufficient time and under such appropriate conditions of temperature, pH, molar ratio, etc., so as to generate a covalent bond between the cell-penetrating peptide and the double-stranded ribonucleic acid molecule. Numerous methods and strategies of conjugation will be readily apparent to one of ordinary skill in the art, as will the conditions required for efficient conjugation.

In one non-limiting embodiment, and by way of example only, one such strategy for conjugation is as follows. In order to generate a disulfide bond between the double-stranded ribonucleic acid molecule and the cell-penetrating peptide, the 3′ or 5′ end of the dsRNA molecule is modified with a thiol group and a nitropyridyl leaving group is manufactured on a cysteine residue of the cell-penetrating peptide. However, any suitable bond may be manufactured according to methods generally and well known in the art (e.g., thioester bonds, thioether bonds, carbamate bonds, etc.). Both the derivatized or modified cell-penetrating peptide and the modified double-stranded ribonucleic acid are reconstituted in RNase/DNase sterile water, and then added to each other in amounts appropriate for conjugation, e.g., equimolar amounts. The conjugation mixture is then incubated for 15 minutes at 65° C., followed by 60 minutes at 37° C., and then stored at 4° C. Linkage can be checked by running the vector-linked siRNA and an aliquot that has been reduced with DTT on a 15% non-denaturing PAGE. siRNA can then be visualized with SyBrGreen.

### 5.2 Delivery of the Cell-Permaeable Complex to Cells

The present invention provides methods of administering a cell-permeable complex to cells of the central nervous system. Methods of the present invention comprise, but are not limited to, contacting a cell of the central nervous system with the cell-permeable complex, thereby reducing the concentration of a target mRNA in the cell and reducing the level of function protein encoded by the target mRNA in the cell.

The present invention provides for a method of admistering the cell-permeable complex to a subject wherein the cell-permeable complex traverses the blood brain barrier, and is widely dispered through the central nervous system of a treated individual.

The present invention further provides for delivery of the cell-permeable complex in vivo to a living organism, for example, but not limited to, a human, rat, mouse, horse, cat, dog, or non-human primate. In a non-limiting embodiment, the administration may be by any procedure known in the art, including but not limited to, oral, parenteral, rectal, intradermal, transdermal or topical administration. To facilitate delivery, the cell-permeable complex of the present invention may be formulated in various compositions with a pharmaceutically acceptable carrier, excipient or diluent, wherein the pharmaceutically acceptable carrier, excipient or diluent of choice does not adversely affect the biological activity of the cell-permeable complex, or the recipient of the composition.

In a preferred non-limiting embodiment of the invention, the membrane-permeable complex is administered through convection-enhanced delivery. Specifically, the cell-permeable complex is administered through convection-enhanced microinfusion, for example, intracerebral clysis (ICC), to the central nervous system (Bruce et al., 2000, Neurosurgery 46(3): 683-691).

In another non-limiting embodiment, delivery of the cell-permeable complex to a cell via ICC has the effect of reducing the level of function protein in a cell encoded by an mRNA targeted by the cell-permeable complex.

In non-limiting embodiments of the invention, ICC delivers the cell-permeable complex by inducing a positive-pressure to distribute the membrane-permeable complex through convection (Bruce et al., 2000, Neurosurgery 46(3):683-691; Bobo et al., 1994, Proc. Natl. Acad. Sci. U.S.A., 91:2076-2080; Broaddus et al., 1998, J. Neurosurg. 88:734-742; Chen et al., 1999, J. Neurosurg. 90:315-320; Lieberman et al., 1995, J. Neurosurg. 82:1021-1029; Morrison et al., 1994, Am. J. Physiol. 266:R292-R305; Zirzow et al., 1999, Neurochem. Res. 24:301-305). According to the present invention, ICC involves the placement of a catheter or cannula into the brain and/or the tumor, and the use of a pump to produce a pressure gradient between the infusion site and the surrounding parenchyma, which distributes the membrane-permeable complex through the interstitial space. Distribution of the cell-permeable complex can be controlled by alterations of the infusion volume and/or rate (Chen et al., 1999, J. Neurosurg 90:315-320).

In a non-limiting embodiment, the methods of the invention are effective to reduce the protein level in a cell encoded by a target mRNA by between 1 and 100%, more preferably between 10 and 95%, and most preferably between 75 and 90%.

The cell-permeable complex can be administered, for example, but not limited to, through a cannula inserted at a depth within the brain. In one non-limiting embodiment, the cannula is inserted to a depth of between 0.1 and 50 mm beneath the surface of the brain, more preferably between 1 and 20 mm beneath the surface of the brain, more preferably between 2 and 10 mm beneath the surface of the brain, and most preferably between 3 and 5 mm beneath the surface of the brain.

In a non-limiting embodiment, the cell-permeable complex is administered to the central nervous system in a solution of between 0.1 and 75% albumin, more preferably between 5 and 50% albumin, more preferably between 10 and 30% albumin, and most preferably 25% albumin. Solutions that are effective for ICC administration are known to those in the art, and a skilled artisan may alter them accordingly when admistering the cell-permeable complex.

In a non-limiting embodiment, the rate at which the cell-permeable complex is administered via ICC to the central nervous system is between 0.01 and 10 μl/min, more preferably between 0.1 and 8 μl/min, more preferably between 0.5 and 5 μl/min., and most preferably 4 μl/min.

In a non-limiting embodiment of the invention, the cell-permeable complex is administered via ICC for a time period of between 1 and 200 min, or between 1 and 100 hours. In one particular non-limiting embodiment of the invention, ICC may be administered for about 100 hours to administer a total of 20 ml

According to the present invention, the cell-permeable complex is contacted with a cell of the central nervous system of an individual to be treated under such conditions of concentration, temperature and pH, etc., and for a sufficient time, to result in delivery of the complex into the cell effective to reduce the concentration of target mRNA, and its encoded protein, in the cell. Specific protocols using the cell-permeable complex of the present invention will vary according to cell type, passage number, cell-penetrating peptide used, etc., but will be readily apparent to one of ordinary skill in the art.

In one non-limiting embodiment, the cell-permeable complex is administered via ICC to an individual, wherein the cell-permeable complex is administered at a concentration of between 1 and 500 μM, more preferably between 10 and 200 μM, more preferably between 20 and f 100 μM, and most preferably 80 μM. Methods and pharmacological carriers suitable for ICC are known by those skilled in the art (Bruce et al., 2000, Neurosurgery 46(3):683-691; Bobo et al., 1994, Proc. Natl. Acad. Sci. U.S.A., 91:2076-2080; Broaddus et al., 1998, J. Neurosurg. 88:734-742; Chen et al., 1999, J. Neurosurg. 90:315-320; Lieberman et al., 1995, J. Neurosurg. 82:1021-1029; Morrison et al., 1994, Am. J. Physiol. 266:R292-R305; Zirzow et al., 1999, Neurochem. Res. 24:301-305).

In one, non-limiting embodiment, the cell-permeable complex is administered via ICC in a total volume efficient to inhibit expression of a target mRNA, where the total volume of administered is between the about 0.1 and 500 μl, or between 0.5 ml and 50 ml.

### 5.3 Conditions to be Treated

### 5.3.1 Inhibition of Tumor Growth

In non-limiting embodiments of the invention, the methods of the present invention can be used to inhibit the growth of tumors which occur in the central nervous system including, but not limited to, gliomas, astrogliomas, chordomas, craniopharyngiomas, medulloblastomas, meningiomas, pineal tumors, pituitary adenomas, primitive neuroectodermal tumors, schwannomas, and vascular tumors such as hemangioblastoma.

A glioma is a type of primary central nervous system (CNS) tumor that arises from glial cells. The most common site of involvement of a glioma is the brain, but they can also affect the spinal cord, or any other part of the CNS, such as the optic nerves. Gliomas usually recur within 2 cm of the original resection margin (Barker et al., 1998, Neurosurgery 42:709-723), and microscopic invasion into normal brain tissue may occur up to 4 cm beyond the tumor margin (Silbergeld et al., 1997, J. Neurosyrg. 86:525-531). Successful therapy for patients with gliomas must target brain tissue into which the tumor has invaded grossly as well as microscopically.

The present invention provides for a method of inhibiting the growth of a tumor (and/or promoting the death of tumor cells) in the central nervous system of a subject, comprising administering, to the central nervous system of the subject, using compositions and methods as described herein, preferably using a method that provides convection-enhanced delivery such as, but not limited to, clysis, an effective amount of a cell-permeable complex effective in inhibiting expression of a target protein, where the target protein inhibits apoptosis (thereby producing a pro-apoptotic effect). In non-limiting embodiments of the invention, the target protein is selected from the group consisting of XIAP, cIAP1 and cIAP2. Reduction of target protein expression as a result of administration of cell-permeable complex may be by at least about 10 percent, by at least about 20 percent, by at least about 30 percent, by at least about 40 percent or by at least about 50 percent.

X-chromosome-linked Inhibitor of Apoptosis Protein (XIAP) is the most potent member of the inhibitor of apoptosis family of proteins (IAP). XIAP prevents the induction of apoptosis normally induced by activated transmembrane death receptors, and confers tumour resistance to irradiation and chemotherapy. XIAP blocks apoptosis by binding to and inhibiting Caspases-3, -7 and -9; proteins necessary for transduction of the apoptotic signal. Furthermore, XIAP has been implicated in tumor development. (Roa et al., 2003, Clin Invest Med 26:231-242).

In a non-limiting embodiment of the invention, administration of the cell-permeable complex increases the activity of pro-apoptotic proteins, for example, but not limited to, Caspase-3, Caspase-7, and Caspase-9.

In a further non-limiting embodiment, the cell-permeable complex is administered to an individual in need of treatment, for example, an individual experiencing tumor growth, or suspected to be at risk for tumor growth, in the central nervous system.

In another non-limiting embodiment of the invention, the cell-permeable complex is administered via ICC, wherein the cell-permeable complex reduces the level of target mRNA to between 1 and 100%, more preferably to between 5 and 80%, more preferably to between 10 and 50%, and most preferably to 20% of the mRNA level prior to treatment. In a further non-limiting embodiment of the invention, the decrease in target mRNA level in a cell subsequently results in a decrease in the level of functional target protein encoded by the target mRNA as compared to untreated cells.

The invention further provides for administration of the cell-permeable complex in or near the tumor, or if the tumor is excised surgically, in the tumor bed, for example by ICC.

### 5.3.2 Ischemia

An ischemic event, such as, for example, a stroke, also known as cerebrovascular accident or a cerebral infarction, is a sudden loss of neuronal function due to a disturbance in cerebral blood flow. This disturbance in perfusion is commonly arterial, but can also be venous. As a result, the part of the brain with the disturbed blood flow will no longer receives adequate oxygen and nutrients. This initates an ischemic cascade which causes brain cells to die or be seriously damaged, impairing local brain function. Upregulation of cell death genes, for example, but not limited to, members of the Caspase family, contribute to the intiation of cell death, or apoptosis, in these blood starved cells. Similarly, hypoxia, or a reduction in oxygen levels available to cells, such as, for example, in hypoxaemia conditions (low blood oxygen content) may also result in death of brain cells.

A cerebral infarction can cause permanent neurological damage or even death if not promptly diagnosed and treated. Factors that increase the likelihood of a cerebral infarction include advanced age, hypertension (high blood pressure), diabetes mellitus, high cholesterol, and cigarette smoking.

An area of the central nervous system may become ischemic as a result of a sudden occlusive event (a classic thrombotic stroke) or a more gradual process, for example partial occlusion of a carotid artery caused by atherosclerosis. Cells of the central nervous system may be subjected to different levels of ischemia depending upon their location relative to a compromised blood vessel; the brain utilizes collateral circulation to protect its most vital areas. Thus, even where certain neurons are damaged beyond rescue, neurons close by may be saveable. Moreover, in conditions where the development of ischemia is transient or insidious, a substantial proportion of cells may be rescued.

The present invention provides methods for treating a variety of ischemic disorders of the central nervous system, including cerebral and spinal infarction, transient ischemic attack, multi-infarct dementia, and ischemic injury which may be caused by trauma (contusion, swelling, or a foreign body), and/or ischemic injury occurring as a result of a hemorrhagic event or a rise in intracerebral pressure from another cause.

The present invention provides for methods of inhibiting or decreasing ischemic damage to the central nervous system, comprising delivering, to the central nervous system, a cell-permeable complex as described herein.

The present invention provides for methods of reducing the expression of proteins implicated as causal in models of neuronal cell death in the context of ischemia. The present invention provides for a method of treating ischemic conditions and inhibiting cell death associated with ischemia in the central nervous system of a subject comprising administering, to the central nervous system of the subject, using compositions and methods as described herein, preferably using a method that provides convection-enhanced delivery such as, but not limited to, intracerebral clysis, an effective amount of a cell-permeable complex effective in inhibiting expression of a target protein, where the target protein promotes apoptosis (that is to say, is pro-apoptotic, thereby inhibiting apoptosis). In non-limiting embodiments of the invention, the target protein is selected from the group consisting of caspase 2, caspase 3, caspase 6, caspase 7, caspase 8, caspase 9, PIDD, RAIDD, and NNOS. Reduction of target protein expression as a result of administration of cell-permeable complex may be by at least about 10 percent, by at least about 20 percent, by at least about 30 percent, by at least about 40 percent or by at least about 50 percent.

For example, and not by means of limitation, Caspase-3 is an effector Caspase which has been implicated as causal in many models of neuronal death. The initial report of Caspase-3 null mice showed overgrowth of the brain and perinatal lethality (Kuida et al., 1996, Nature 384:368-372). However, Caspase-3 null mice on a C57/B16 background are not embryonic lethal and show protection against middle cerebral artery occlusion (MCAo), a method known in the art to model a cerebral infarction, or stroke (Le et al., 2002, Proc Natl Acad Sci USA 99:15188-15193).

In a specific non-limiting embodiment, the present invention provides for methods of reducing the level of functional Caspase-3 in a cell by contacting the cell with a cell-permeable complex in an amount effective to reduce Caspase-3 mRNA. According to the invention, the cell-permeable complex comprises a cell-penetrating peptide, for example, but not limited to, Penetratin-1, operably linked to an siRNA that binds Caspase-3 mRNA and targets it for degradation.

In another non-limiting embodiment of the invention, the cell-permeable complex is administered to an individual in need of treatment, for example, an individual experiencing, or suspected to be at risk for, an acute or chronic restriction of oxygen or blood supply to the central nervous system.

In one non-limiting embodiment of the invention, the cell-permeable complex is administered via ICC, wherein the cell-permeable complex reduces the level of target mRNA to between 1 and 100%, more preferably to between 5 and 80%, more preferably to between 10 and 50%, and most preferably to 10% of the level of mRNA in untreated cells. In a further non-limiting embodiment of the invention, the decrease in target mRNA levels in a cell subsequently results in a decrease in the level of functional target protein encoded by the target mRNA as compared to untreated cells.

The present invention provides for delivery of a cell-permeable complex to cells of the central nervous system damaged by ischemia, or suspected to be in danger of being damaged, by ischemia. In a non-limiting embodiment, the cell-permeable complex is administered, for example, but not limited to, via ICC instillation, such that the cell-permeable complex enters cells of the central nervous system damaged, or suspected to be in danger of being damaged, by ischemia. In a non-limiting embodiment, the cell-permeable complex can be delivered to any cells of the central nervous system, for example, but not limited to, cells of the hippocampus, thalamus, striatum, cerebellum, medulla, pons, hypothalamus, cerebral cortex, and/or spinal cord.

In a further non-limiting embodiment, the present invention provides for a method of inhibiting inflammation in the central nervous system of a subject comprising administering, to the central nervous system of the subject, using compositions and methods as described herein, preferably using a method that provides convection-enhanced delivery such as, but not limited to, intracerebral clysis, an effective amount of a cell-permeable complex effective in inhibiting expression of a target protein, where the target protein promotes inflammation (thereby inhibiting inflammation). In non-limiting embodiments of the invention, the target protein is caspase 1.

### 6 Example 1

### Generation of siRNA Sequences

siRNA sequences: siRNA were designed to target various mRNAs. A general strategy for designing siRNAs comprises beginning with an AUG stop codon and then scanning the length of the desired cDNA target for AA dinucleotide sequences. The 3′ 19 nucleotides adjacent to the AA sequences were recorded as potential siRNA target sites. The potential target sites were then compared to the appropriate genome database, so that any target sequences that have significant homology to non-target genes could be discarded. Multiple target sequences along the length of the gene were located, so that target sequences were derived from the 3′, 5′ and medial portions of the mRNA. Negative control siRNAs were generated using the same nucleotide composition as the subject siRNA, but scrambled and checked so as to lack sequence homology to any genes of the cells being transfected. (Elbashir, S. M., et al., 2001, Nature, 411, 494-498; Ambion siRNA Design Protocol, at www.ambion.com).

Target sequences were 21 bases long, beginning with AA. siRNA which bind the target sequences were modified with a thiol group at the 5 C6 carbon on one strand. Custom siRNAs were generated on order from Dharmacon Research, Inc., Lafayette, Colo. Other sources for custom siRNA preparation include Xeragon Oligonucleotides, Huntsville, Ala. and Ambion of Austin, Tex. Alternatively, siRNAs can be chemically synthesized using ribonucleoside phosphoramidites and a DNA/RNA synthesizer. Target sequences that the siRNA were designed to are as follows: Caspase-3 (5′ thiol on sense strand) AGC CGA AAC UCU UCA UCA U (SEQ ID NO:1) (GenBank accession number BC038825, initiation at base 111, target bases 569-589; GenBank accession number NM_{—}012922, initiation at base 57, target bases 517-535); Caspase-7 (5′ thiol on sense strand) GAU GCA GGA UCU GCU UAG A (SEQ ID NO:2) (GenBank accession number BC070936, inititation at base 3, target bases 356-374); Caspase-8 (5′ thiol on antisense, 5′ FITC on sense): AAG CAC AGA GAG AAG AAU GAG (SEQ ID NO:3) (GenBank Accession No. BC006737, initiation at base 336, target bases 878-898); Caspase-8 (5′ thiol on antisense): AAG AAG CAG GAG ACC AUC GAG (SEQ ID NO:4) (GenBank Accession No. BC006737, initiation at base 336, target bases 432-452); Caspase-9 (5′ thiol on antisense): AAG GCA CCC UGG CUU CAC UCU (SEQ ID NO:6) (GenBank Accession No. NM015733, initiation at base 244, target bases 488-508); XIAP (5′ thiol on sense strand) CUG GAC AGG UUG UAG AUA U (SEQ ID NO: 8) (GenBank Accession Number NM_{—}009688, initiation at base 672, target bases 1099-1117, GenBank Accession Number AB033366, initiation at base 330, target bases 757-775); Caspase-1 GAA GGC CCA UAU AGA GAA A (SEQ ID NO: 16) (GenBank accession number BC008152, initiation at base 201, target bases 1151-1169; GenBank accession number NM_{—}012762, initiation at base 1, target bases 951-969); Caspase-2 GCC AUG CAC UCC UGA GUU U (SEQ ID NO: 17) (GenBank accession number NM_{—}007610, initiation at base 86, target bases 616-634; GenBank accession number NM_{—}022522, initiation at base 7, target bases 537-555); Caspase-6 GGG UAU UAC UCU CAC CGA GA (SEQ ID NO:18) (GenBank accession number BC002022, initiation at base 57, target bases 645-665; GenBank accession number BC078785, initiation at base 187, target bases 778-797); CCA CAU UCA AGAAAU CAA G (SEQ ID NO:22) (GenBank accession number MMAJ4740, initiation at base 105, target bases 221-238; and GenBank accession number XM_{—}001080418, initiation at base 112, target bases 228-245); NNOS CCU CGU GAA UGC ACU CAU U (SEQ ID NO:23) (GenBank accession number NM_{—}008712, initiation at base 79, target bases 2427-2445; GenBank accession number: X59949, initiation at base 349, target bases 3474-3492.); and PIDD CCU GGG UGA UGC AGA AAC U (SEQ ID NO:21) (GenBank accession number AF274973, initiation at base 79, target bases 2427-2445).

### 7 Example 2

### Preparation of Cell-Permeable Complex

Penetratin-1 cell-penetrating peptide: Penetratin-1 (mw 2503.93) comprising the peptide sequence RQIKIWFQNRRMKWKK (SEQ ID NO:7) (QBiogene, Inc., Carlsbad, Calif.) was reconstituted to 2 mg/ml in RNase/DNase sterile water (0.8 mM). siRNA (double-stranded, annealed, and synthesized with a 5′-thiol group on the sense or antisense strand) was reconstituted to 88 μM in RNase-/DNase-free sterile water. To link the Penetratin-1 to the siRNA, 25 μl of Penetratin-1 were added to 225 μl of the diluted oligo, for total volume of 250 μl. This mixture was incubated for 15 min at 65° C., followed by 60 min at 37° C., then stored at 4° C. Alternatively, where only small amounts of the mixture are required, these were aliquoted and stored at −80° C. Linkage was be checked by running the vector-linked siRNA and an aliquot that had been reduced with DTT on a 15% non-denaturing PAGE. siRNA was visualized with SyBrGreen (Molecular Probes, Eugene, Oreg.). In an alternative method of coupling the siRNA and the Penetratin-1, siRNA duplexes with a 5′ thiol on the sense strand were synthesized and HPLC purified (Dharmacon, Lafayette, Colo.). Annealed siRNA duplexes were resuspended in buffer provided by manufacturer, treated with an equimolar ratio of Penetratin-1 (Q-Biogene, Carlsbad, Calif.) added and incubated at 65° C. for 5 minutes, followed by 37° C. for 1 hour. The yields of the reactions were estimated by SDS-PAGE using Coomassie blue staining. Efficacy of each Pen1-siRNA construct for knock-down of target was determined in hippocampal neuronal cultures that are routinely grown by methods known in the art, using measures of RNA and protein expression.

### 8. Example 3

### Transfection Efficiency of Cells in Culture

Transfection efficiencies of neuronal cells are generally low. To increase efficiency of delivery of siRNA to neuronal cells, a cell-permeable complex was created in which an siRNA molecule was linked to a cell-penetrating peptide. Specifically, either of the sense or antisense strand of each siRNA was modified at its 5′ end with a thiol group by methods known in the art, and covalently bonded via a disulfide bond with a Penetratin-1 peptide having a pyridyl disulfide function at its terminal end. The cell-permeable complex was incubated with sympathetic neuron cultures, and efficiency of transport into the cells was visualized immunohistochemcally.

Primary mouse sympathetic neuron cell cultures: Cell cultures were prepared as follows. Sympathetic neuron cultures were prepared from 1-day-old wild-type mouse pups, as previously described (Troy, et al., 2000, J. Neurosci., 20, 1386-1392). Cultures were grown in 24-well collagen-coated dishes for survival experiments, and in 6-well collagen-coated dishes for RNA and protein extraction in RPMI 1640 medium (Omega Scientific, Tarzana, Calif.; ATCC, Manassas, Va.) plus 10% horse serum with mouse NGF (100 ng/ml). One day following plating, uridine and 5-fluorodeoxyuridine (10 μM each) were added to the cultures, and left for three days to eliminate non-neuronal cells. (Less than 1% non-neuronal cells remain after 3 days.)
Primary rat hippocampal neuron cell cultures: Hippocampi were dissected from embryonic day 18 (E18) rat fetuses, dissociated by trituration in serum-free medium, plated on 0.1 mg/ml poly-D-lysine-coated tissue culture wells or plastic Lab-Tek slide wells, and maintained in a serum-free environment. The medium consisted of a 1:1 mixture of Eagle's MEM and Ham's F12 (Gibco, Gaithersburg, Md.) supplemented with glucose (6 mg/ml), putrescine (60 μM), progesterone (20 nM), transferrin (100 μg/ml), selenium (30 nM), penicillin (0.5 U/ml), and streptomycin (0.5 μg/ml) (Sigma, St. Louis, Mo.). In all experiments, neurons were cultured for 4-5 days before treatment. Cultures contained <2% glial cells, as confirmed by staining for glial markers.

Immunocytochemistry was performed according to the following protocol. Cultured cells were fixed with 4% paraformaldehyde, exposed to primary antibodies at room temp for 1.5 h, washed with PBS, exposed to the appropriate fluorescent secondary antibodies for 1 h at room temperature, followed by Hoechst stain for 15 min at room temperature, and then analyzed with a Nikon fluorescent microscope. For uptake studies, living cultures were treated with FITC-siRNA, and analyzed with a Perkin-Elmer Spinning Disc confocal imaging system mounted on a Nikon inverted microscope.

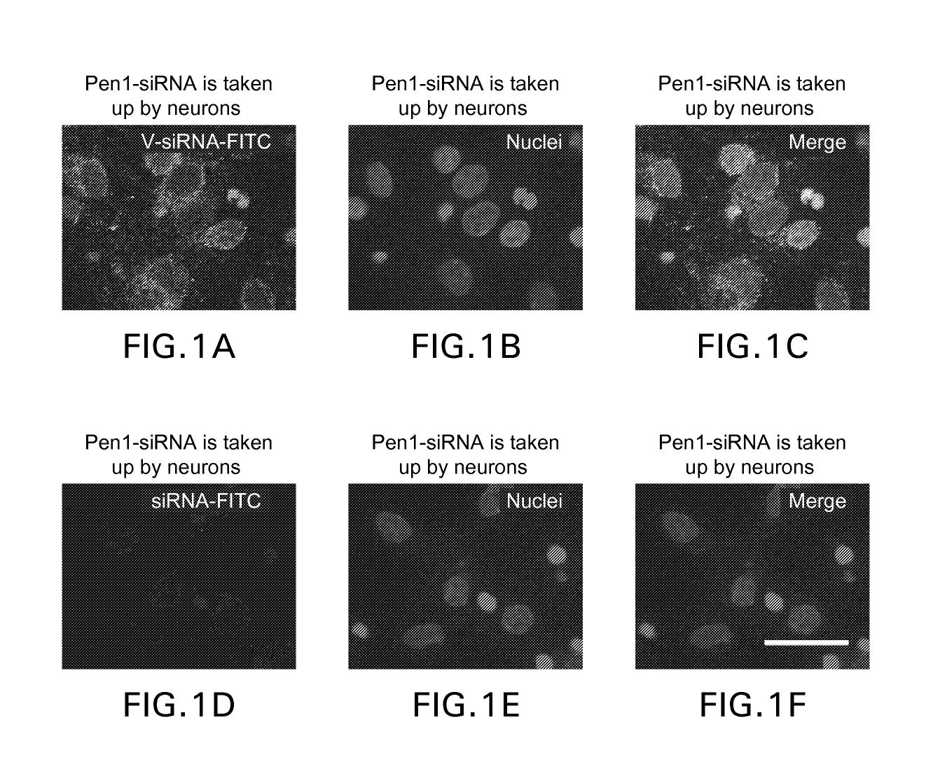

siRNA labeled with FITC was linked to the Penetratin-1 peptide, and applied to cultured rat hippocampal neurons. FITC was visualized with confocal microscopy. Uptake was rapid, within minutes of application of siRNA, the complex could be detected in the cells (FIG. 1A-C). Cultured hippocampal neurons treated with siRNA labeled with FITC and not linked to Penetratin-1 was not readily taken up by the cells, and was not readily detectable (FIG. 1D-F).

### 9. Example 4

### Specific Inhibition of mRNA's In Vitro

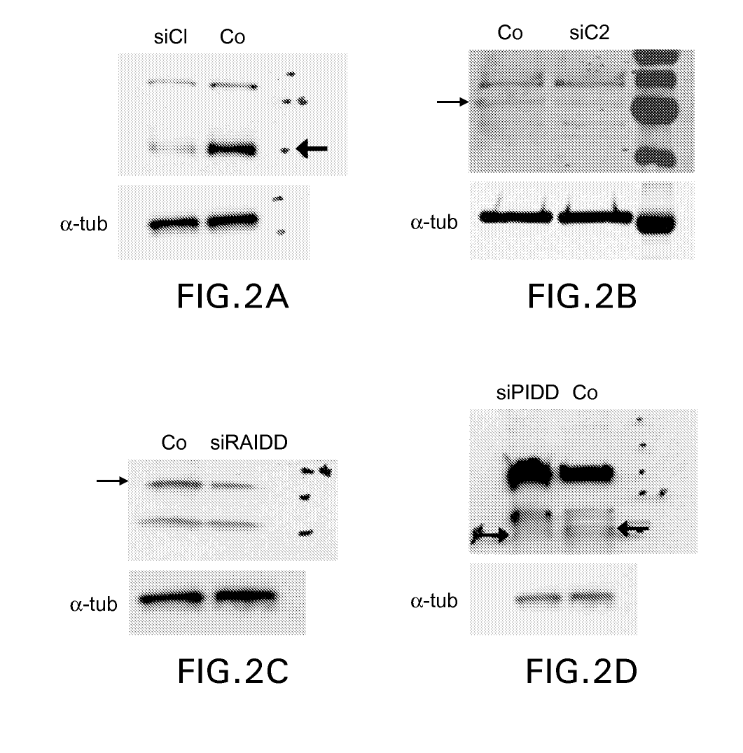

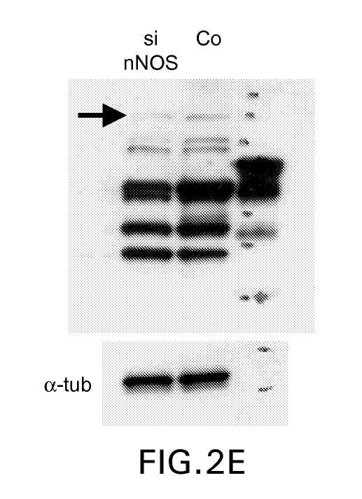

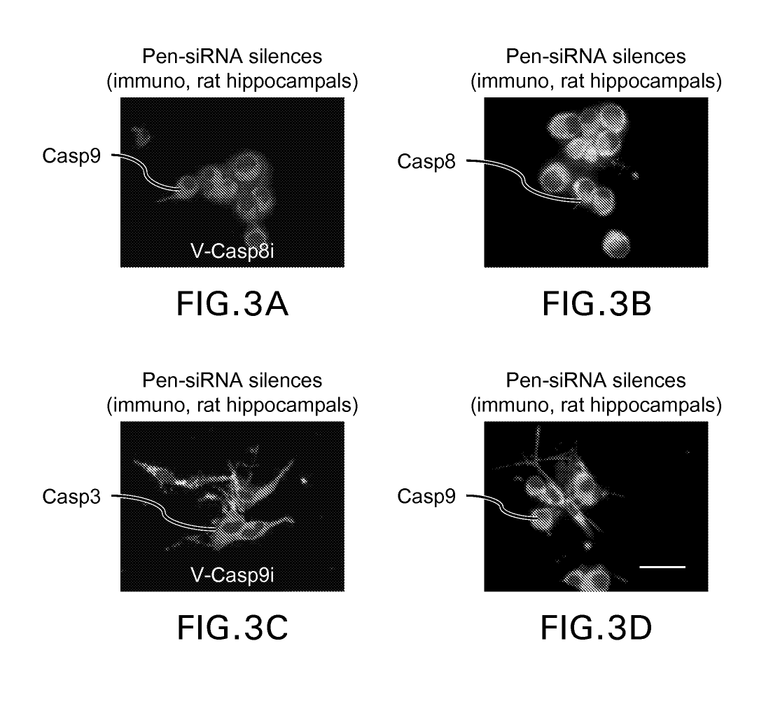

Cultured mouse sympathetic neurons: Cell cultures were prepared as follows. Sympathetic neuron cultures were prepared from 1-day-old wild-type, as previously described (Troy, et al., 2000, J. Neurosci., 20, 1386-1392). Cultures were grown in 24-well collagen-coated dishes for survival experiments, and in 6-well collagen-coated dishes for RNA and protein extraction in RPMI 1640 medium (Omega Scientific, Tarzana, Calif.; ATCC, Manassas, Va.) plus 10% horse serum with mouse NGF (100 ng/ml). One day following plating, uridine and 5-fluorodeoxyuridine (10 μM each) were added to the cultures, and left for three days to eliminate non-neuronal cells. (Less than 1% non-neuronal cells remain after 3 days).
Primary rat hippocampal cell culture: For Pen1-siCasp3 and Pen1-siXIAP experiments, primary rat hippocampal cells were prepared as follows. Hippocampi were dissected from embryonic day 18 (E18) rat fetuses, dissociated by trituration in serum-free medium, plated on 0.1 mg/ml poly-D-lysine-coated tissue culture wells or plastic Lab-Tek slide wells, and maintained in a serum-free environment. The medium consisted of a 1:1 mixture of Eagle's MEM and Ham's F12 (Gibco, Gaithersburg, Md.) supplemented with glucose (6 mg/ml), putrescine (60 μM), progesterone (20 nM), transferrin (100 μg/ml), selenium (30 nM), penicillin (0.5 U/ml), and streptomycin (0.5 μg/ml) (Sigma, St. Louis, Mo.). In all experiments, neurons were cultured for 4-5 days before treatment. Cultures contained <2% glial cells, as confirmed by staining for glial markers.
Immunocytochemistry: Immunocytochemistry in all three series of experiments was performed according to the following protocol. Cultured cells were fixed with 4% paraformaldehyde, exposed to primary antibodies for Caspase-3, -8, -9, or XIAP at room temp for 1.5 h, washed with PBS, exposed to the appropriate fluorescent secondary antibodies for 1 h at room temperature, followed by Hoechst stain for 15 min at room temperature, and then analyzed with a Nikon fluorescent microscope.
RealTime Quantitative PCR: Primers were designed to amplify a 300-400 base piece of the gene of interest. Optimal primer size was 15-20 bases. cDNA from brains were added to a reaction mix (PCR ready-to-go beads, Amersham Pharmaceuticals, with SYBR Green, Molecular Probes) together with appropriate primers at 0.5 μM each. Levels of transcripts were analyzed using the Cepheid SmartCycler (Fisher) following the manufacturer's specifications. Real time fluorescence of SYBR green indicated that double-stranded DNA was measured. Melting curve analysis was used for each protocol to characterize and identify the specific amplicon. In each case quantification was made from the linear portion of the amplication curve. Alpha-tubulin was used to normalize input cDNA.
Caspase-1, Caspase-2, Caspase-6, RAIDD, PIDD and NNOS: siRNA were designed for Caspase-1, Caspase-2, Caspase-6, RAIDD, PIDD, and NNOS. The siRNA were then linked to the Penetratin-1 peptide. Cultured rat hippocampal neurons were treated with each of these constructs. Cultures were grown for one day, then harvested for protein and mRNA analysis. Expression levels of protein encoded my the targeted mRNA was analyzed by Western Blotting. mRNA was analyzed with RealTime Quantitative PCR. Expression of the targeted mRNA was inhibited in all of the cultured cells as compared to untreated control cells (FIG. 2A-E). mRNA levels were decreased by 80% (Caspase-1), 70% (Caspase-2), 60% (Caspase-6), 80% (RAIDD), and 90% (NNOS), as compared to untreated cells.
Caspase-8 and Caspase-9: siRNA were designed for two members of the Caspase family of death proteases, Caspase-8 and Caspase-9, and linked to the Penetratin-1 peptide. Cultured mouse sympathetic neurons were treated with each of these constructs. Cultures were grown for one day, fixed and immunostained for Caspase-8 (FIG. 3A-B) or Caspase-9 (FIG. 3C-D), together with Hoechst stain, and then visualized with fluorescent microscopy. Expression of the targeted Caspase (Caspase-8 or Caspase-9) was inhibited in all of the cultured cells. Expression of non-targeted Caspases was not changed.

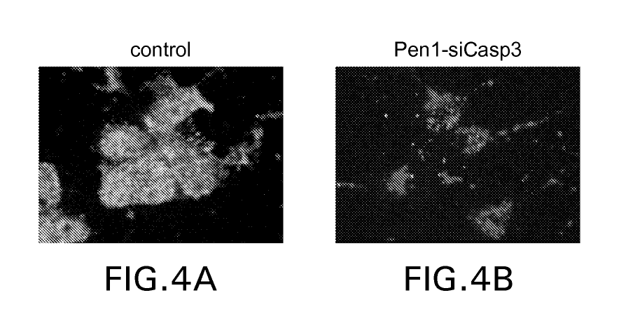

Caspase-3: Pen1-siRNA was designed to target the Caspase-3 that is conserved in rats and mice. The sequence was synthesized as a 21 base double-stranded RNA with a thiol-modification on the 5′ end of the sense strand, as previously described. The sequence was linked to Pen1 and tested for efficacy in primary rat hippocampal cultures. Using RealTime Quantitaive PCR we found that Pen1-siCaspase-3 provided 90% reduction of the Caspase-3 mRNA after one day treatment. After one day treatment there was substantial down-regulation of the targeted proteins (FIG. 4B), as visualized by immunocytochemistry. compared to control cells not treated with Pen1-siCasp3 (FIG. 4A).

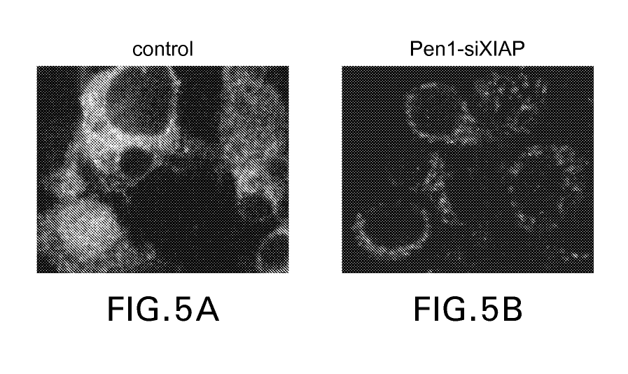

XIAP: Pen1-siRNA was designed to target the XIAP ORF region that is conserved in rats and mice. The sequence was synthesized as a 21 base double-stranded RNA with a thiol-modification on the 5′ end of the sense strand. The sequence was linked to Pen1 and tested for efficacy in primary rat hippocampal cultures. Using RealTime PCR it was found that Pen1-siXIAP provided 80% reduction of the XIAP mRNA after one day treatment. After one day of treatment there was substantial down-regulation of the targeted proteins (FIG. 5B), as visualized by immunocytochemistry, compared to control cells not treated with Pen1-siXIAP (FIG. 5A).

### 10. Example 5

### Optimal Delivery Via ICC

Intracerebral Clysis in Rats: Adult male Wistar rats (250-300 g) were anesthetized via rat anesthesia mask for stereotactic instruments (Stoelting) and placed in a stereotactic frame. The scalp was shaved and the skin was prepped with iodine solution, and infused with 0.25 ml of 0.25% bupivicaine solution. A 1.0-1.5 cm incision was made in the midline of the scalp to expose the bregma. A 1 mm burrhole was created at the coordinates 1 mm anterior and 3 mm lateral to the bregma. For the acute stereotactic infusions, a 28 gauge cannula was inserted to a depth of 5 mm below the dura into the caudate nucleus (Bruce et al., 2000, Neurosurgery 46:683-691). Infusion of therapeutic was then instituted. Following infusion, the cannula was removed at a rate of 1 mm/minute, the burrhole was sealed with bone wax, and the skin incision was closed. The animal was returned to the incubator and maintained at normothermia until the completion of the 90 minute post-operative period.

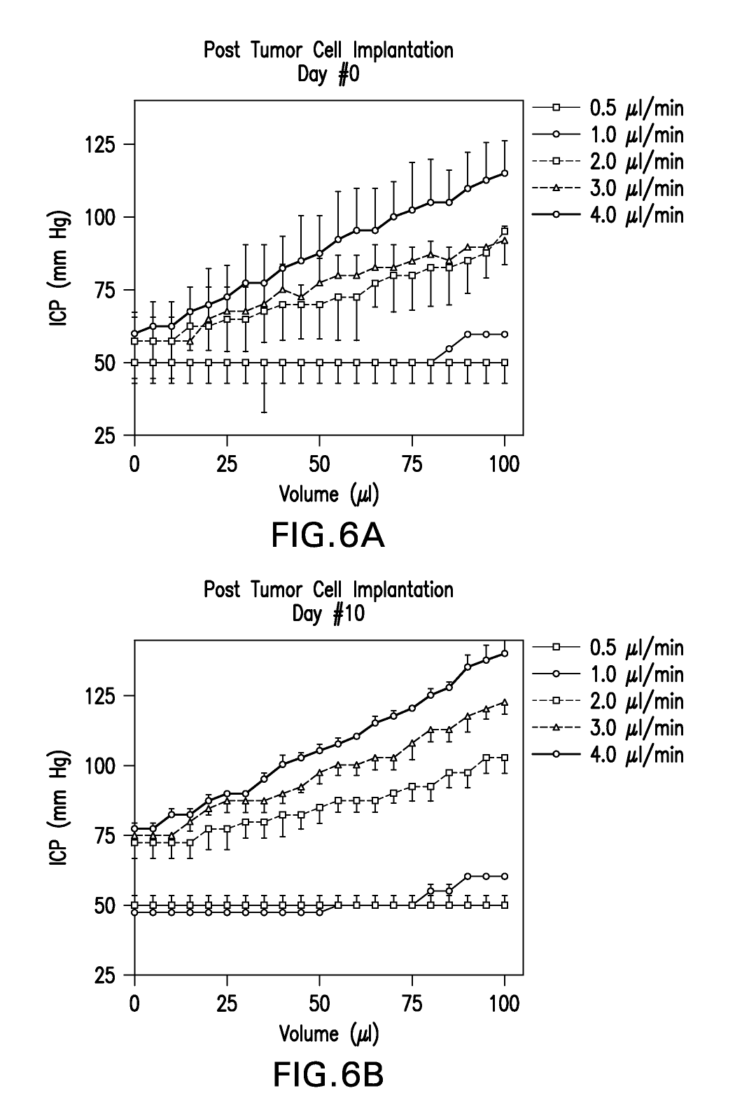

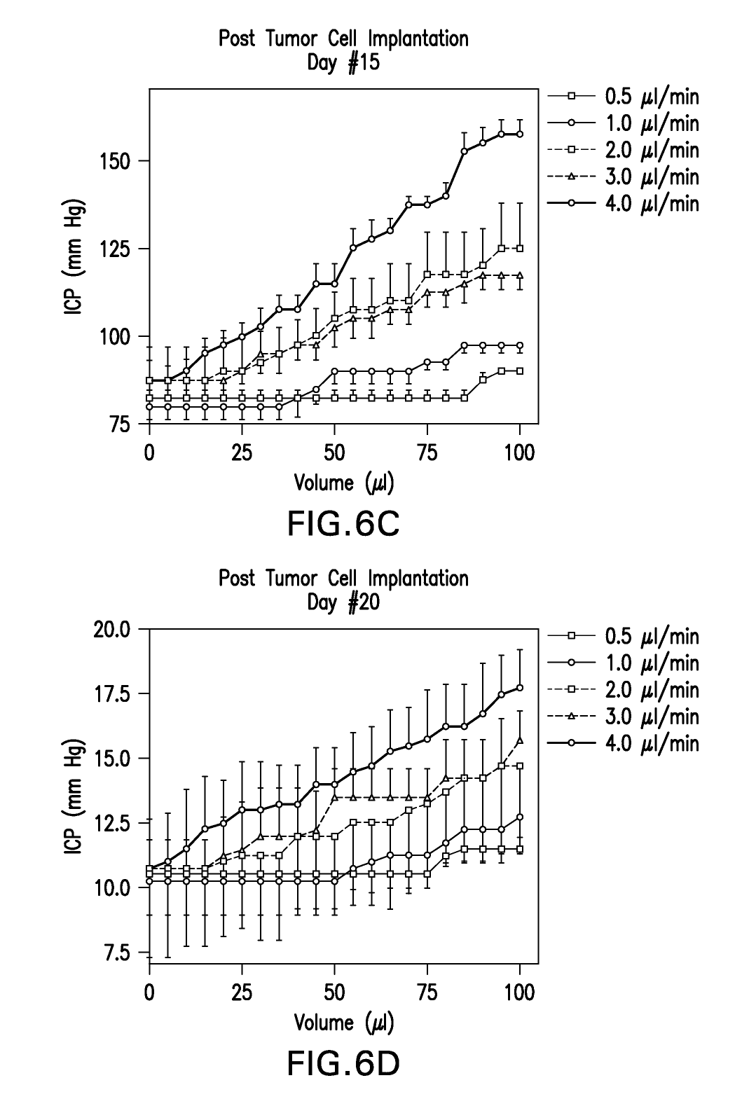

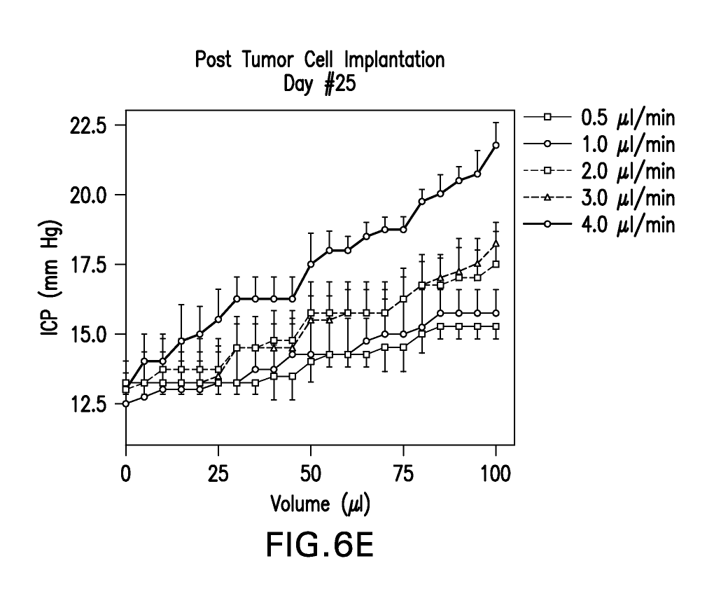

To demonstrate the clinical utility of ICC in rats with space-occupying intracranial masses, the tolerated infusion rates and volumes were determined by measuring ICP via a fiber-optic ICP monitor. In order to optimize delivery parameters and investigate the limitations of ICC delivery, ICP was measured as the flow rates and infusion volumes of a 25% albumin solution was varied. Flow rates were varied between 0.5, 1.0, 2.0, 3.0 and 4.0 μl/min until a final volume of 100 μl was introduced via ICC. Animals were administered ICC following tumor cell implantation on days 0, 10, 15, 20 and 25 post tumor cell implantation. For measuring ICP, the fiber-optic ICP catheter was inserted 3.0 mm below the surface of the brain at a location 3.0 mm posterior to bregma, and 3.0 mm lateral to the midline. ICP changes associated with rates of 0.5 and 1.0 μl/min were significantly smaller than those associated with flow rates of 2.0-4.0 μl/min (FIG. 6).

### 11. Example 6

### Distribution of Compounds Delivered by ICC

Intracerebral Clysis in Rats: Adult male Wistar rats (250-300 g) were anesthetized via rat anesthesia mask for stereotactic instruments (Stoelting) and placed in a stereotactic frame. The scalp was shaved and the skin was prepped with iodine solution, and infused with 0.25 ml of 0.25% bupivicaine solution. A 1.0-1.5 cm incision was made in the midline of the scalp to expose the bregma. A 1 mm burrhole was created at the coordinates 1 mm anterior and 3 mm lateral to the bregma. For the acute stereotactic infusions, a 28 gauge cannula was inserted to a depth of 5 mm below the dura into the caudate nucleus (Bruce et al., 2000, Neurosurgery 46:683-691). Infusion of therapeutic was then instituted. Following infusion, the cannula was removed at a rate of 1 mm/minute, the burrhole was sealed with bone wax, and the skin incision was closed. The animal was returned to the incubator and maintained at normothermia until the completion of the 90 minute post-operative period. Rats were then sacrificed, and brains were section according to methods known by those in the art. FITC was detected with a Nikon fluorescent microscope using a Spot digital camera.

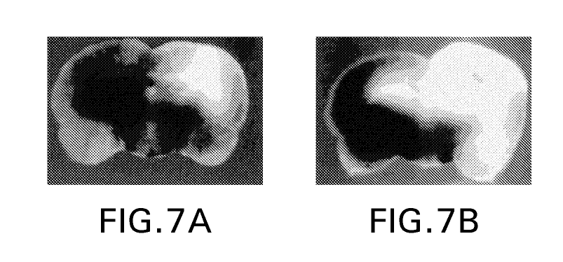

A wide macromolecule distribution is achieved through ICC administration. As shown in FIG. 7A-B, the distribution of FITC-dextran after delivery via ICC shows a macromolecule distribution pattern throughout the hemisphere that was infused. ICC was adminstered at a flow rate of 3.0 μl until a total of volume of 10 μl was infused (FIG. 7A), or 30 μl was infused (FIG. 7B).

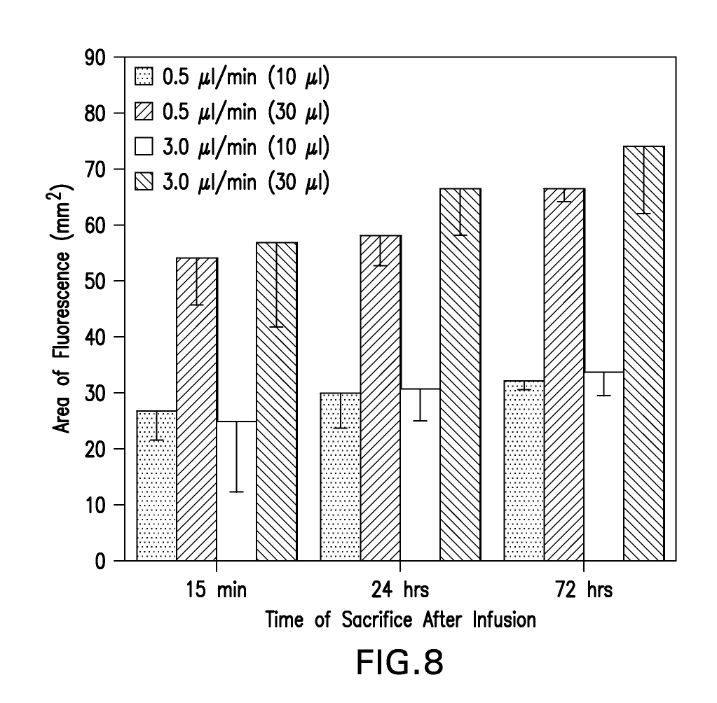

Infusion of compounds delivered by ICC achieved the greatest distribution when the total volume infused increased. Animals infused with a total volume of 30 μl exhibited a wider distribution of infusion than animals infused with a total volume of 10 μl, regardless of whether the infusion rate was 0.5 μl or 3 μl (FIG. 8).

### 12. Example 7

### Pen1-siRNA Delivery to the Rat Brain In Vivo Via ICC

The transduction peptide Penetratin-1 facilitates uptake of siRNA by neurons in culture and has been shown to increase the cerebral tissue uptake of a cargo molecule after intracarotid injection 6 fold. ICC has proved very effective in delivering small molecules to the brain. Delivery of Pen1-siRNA to the brain using ICC was examined to determine efficacy of transfer and toxicity of the delivery method.

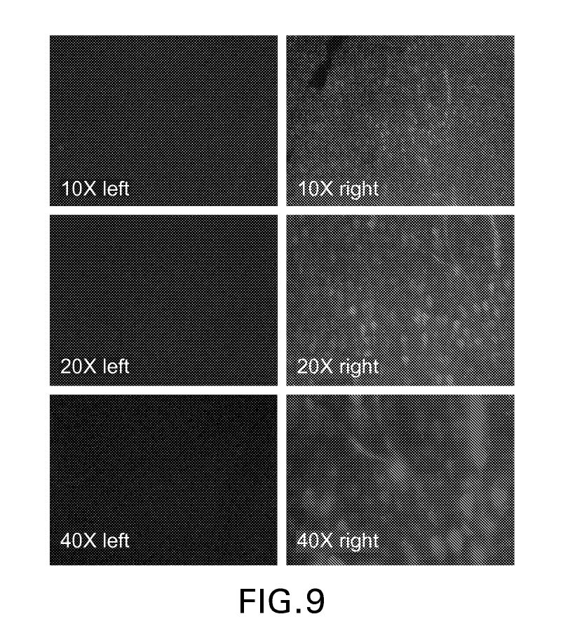

Pen1-siRNA was delivered to the brain in vivo with the ICC method. Rhodamine-labeled Pen1-siRNA (55 μM) was administered to an adult male Wistar rat by ICC delivery to the right side of the brain. The catheter for the ICC was placed 3 mm lateral of the midline, 1 mm anterior to bregma, and 5 mm deep (measured from the outer table of the calvarium). A total of 30 μl was delivered over 60 minutes (at a rate of 0.5 μl/min). The rat was sacrificed 24 hours later and the brain was fixed and sectioned. Sections were imaged without further processing using a Nikon fluorescent microscope with a Spot digital camera. FIG. 9 shows that there is substantial uptake of rhodamine—Pen1-siRNA on the right side of the brain while there is no detectable uptake on the left side. The rhodamine label is on the siRNA and is detected within cells and processes. This experiment indicates that siRNA can be successfully delivered to the cells and retained for at least 24 hours after delivery.

### 13. Example 8

### Half-Life of Pen1-siRNA in Transformed Cells In Vivo

Caspase-3 and XIAP may be targeted with membrane permeable complex to determine the time period over which the interference of the targeted mRNA by the cell-permeable complex.

Pen1-siRNA to Caspase-3 and XIAP may be delivered via ICC or intracarotid injection, and brains assessed for knockdown of targeted gene and protein using in situ hybridization, RealTime PCR, immunocytochemistry and Western blotting. Each treatment group may contain 16 animals at each time point to provide enough animals for analysis. Animals may be sacrificed at 5 h, 24 h and 2 days after Pen1-siRNA delivery. Brains may be prepared for sectioning for in situs and immunocytochemistry, and for RNA extraction and protein extraction. Using RealTime PCR and Western blotting, the expression of the targeted gene product may be examined. Other members of the family (i.e. other Caspases and other IAPs) and unrelated molecules may be examined to determine whether there are off-target effects. For Western the two-color Odyssey detection system may be used which allows detection of mouse and rabbit primary antibodies at the same time, so that loading controls may be done simultaneously with measurements of the protein of interest. Analysis for induction of interferon as a toxic effect of the siRNA may be performed.

Intracarotid injection: For this technique, the anesthesia, exposure, surgical approach, and post-operative care is the same as for the middle cerebral artery occlusion detailed below. To perform the intracarotid injection, either without or after middle cerebral artery occlusion, a modified length of PE-50 catheter may be constructed by heating and stretching over a flame until the outer diameter of the catheter approximates the diameter of a 6-0 nylon monofilament. This flexible catheter may then be inserted into the external carotid stump in the same way that the occluding filament is advanced in the stroke model. The catheter may then advanced into the internal carotid artery a distance of 8 mm from the carotid bifurcation, to rest at the origin of the middle cerebral artery. The total volume of siRNA may then be injected via microinfusion pump over a period of 5 minutes, after which the catheter is removed and animal recovery proceeds as detailed for the stroke model.

### 14. Example 9

### Rat Ischemia Model

Delivery of siRNA to the brain via ICC may provide a method of studying the function of individual proteins and will also provide a potential therapeutic for diseases of the brain. The rodent middle cerebral artery occlusion (MCAo) stroke model (Connolly et al., 1996, Neurosurgery 38:523-532; Ivanova et al., 2002, Proc Natl Acad Sci USA 99:5579-5584), as well as a well-established stroke model in non-human primates (Huang et al., 2000, Stroke 31:3054-3063; D'Ambrosio et al., 2004, Methods Enzymol 386:60-73) may be used to study the effect of Pen1-siRNA on cerebral infarction. These models will be used to determine functional efficacy of the Pen1-siRNA. Multiple molecules have been implicated in the neuronal death that follows MCAo. Caspase-3 null mice on a C57/B16 background have been shown to be partially protected from MCAo (Le et al., 2002, Proc Natl Acad Sci USA 99:15188-15193). Pen1-siRNAi targeted to Caspase-3 mRNA, Pen 1-Casp3, has been characterized.

Rats may be subjected to MCAo in the presence or absence of Pen1-siCasp3, delivered via ICC, with 10 animals in each group. Animals may be examined clinically using the rotarod and foot fault tests and sacrificed at 1, 2, 3, and 7 days after infarct and brains examined for extent of infarct and presence of cleaved Caspase-3. To determine the extent of infarct up to 3 days, the vital dye TTC may be used to stain fresh 1 mm brain slices. For the 7 day time points, brain sections may be stained with H&E to evaluate infarct size. Brain sections may be immunologically stained for cleaved Caspase-3 and labeled for TUNEL to evaluate death.

Rat Ischemia (Stroke) Model: Adult Wistar male rats (250-300 g) may be anesthetized using halothane delivered in a mixture of nitrous oxide (70%) and oxygen (30%) via facemask, and MCAo may be accomplished with a 25 mm 4-0 nylon suture (5 mm silicone rubber tip) occluding the MCA. The occluding suture may be removed after 120 min. To confirm cerebral ischemia, transcranial measurements of cerebral blood flow (CBF) may be made using laser-Doppler flowmetry over the MCA territory (1.5 mm posterior and 5.5 mm lateral to the bregma). Reduction of LDF readings to at least 40% of baseline is defined as adequate CBF drop-off. The degree of functional deficit at 1 hour post-occlusion is scored using a modified 5-point Bederson scale (Bederson et al., 1986, Stroke 17:472-476). Animals with Bederson's scores less than 1 (no deficit) are excluded from analysis. Animals may be weighed, scored on the 5-point Bederson scale, and tested on two well-characterized functional tasks (rotarod, foot-fault tests) on post-op days 1, 3 and 7. Animals may then be sacrificed on days 1, 2, 3 and 7, and histological infarct may be quantified by integrating the volume of infarction determined with either vital dye staining with TTC for days 1 and 2 or, for days 3 and 7, indirectly on multiple hematoxylin & eosin cryostat sliced 10 micron sections (Lin et al., 1993, Stroke 24:117-121). In addition to these cerebral ischemia experiments, sham ischemic surgery may be used to control for the effects of the threading procedure. For sham experiments, the occluding suture may be introduced into the vessel and immediately withdrawn.
Foot-Fault test: Animals may be placed on an elevated wire grid made from test tube racks with openings of 2.8 cm×2.8 cm. With each weight-bearing step, the animal's forefoot is able to slip below the wire grid. The number of slips for each forefoot in forty weight bearing steps is recorded and expressed as a percentage of total steps (Aronowski et al., 1996, J Cereb Blood Flow Metab 16:705-713).
Rotarod Test The rotarod may be utilized for functional outcome analysis at extended time points (Hunter et al., 2000, Neuropharmacology 39:806-816). Animals are subjected to 3 trials on the rotating cylinder daily for 3 days prior to the experiment (pre-training). The amount of time that the animal remains on the rotating cylinder, which accelerates at a constant velocity over 5 minutes, is recorded. These trials are averaged and the mean may be taken as the animal's baseline. On days 3 and 7, animals are subjected to two trials, and the results are averaged. Results may be expressed as a percentage of the baseline score for the animal. The rotarod is reported as a percentage of each animal's baseline (baseline=1.0). The baseline is computed as the average of 3 trials per day over the 3 days of preoperative training.
Immunocytochemistry for stoke animal models: Rats may anesthetized, perfused with 4% paraformaldehyde and ipsilateral and contralateral hemispheres prepared for cryostat. For detection of fluorescent siRNA, sections may be imaged with an upright Nikon fluorescent microscope. For detection of biotinylated Penetratin-1, ABC detection (Vector Labs) may be used. For analysis of target protein expression, sections may be immunostained with antisera to the target protein. After 30 min block with 3% normal goat serum, slides may be incubated with primary antibody overnight, then washed with PBS and incubated with appropriate secondary antibody (goat-anti-rabbit or anti-mouse conjugated with rhodamine or FITC) for 1 hour, followed by three PBS washes and incubated with Hoechst 33342 (1 μg/ml). Samples may be visualized with a Perkin-Elmer Spinning Disc Confocal Imaging System. Adjacent sections may be stained with hematoxylin and eosin to define morphology.
Tunel staining: Adjacent sections may be processed for DNA fragmentation by TdT-mediated dUTP nick end labeling (TUNEL) using an in situ cell death detection kit (Roche Diagnostics GmbH, Mannheim, Germany). In brief, the sections may be dried and permiabilized for 5 minutes with ethanol 95%-acetic acid (2:1) at −20° C. They may then be incubated with a mixture of terminal deoxynucleotidyl transferase and fluorescein-conjugated deoxyuridine triphosphate for 1 hour at 37° C. Sections may be visualized with a Perkin-Elmer Spinning Disc Confocal Imaging System. As a negative control, the enzyme may be omitted in the incubation mixture.

### 15. Example 10

### Mouse Ischemia Model

A stroke may be induced in mice as described previously for rats, in the presence or absence of Pen1-siCasp3. Animals may be examined clinically at 1 day using the 4 point neurologic scale (Connolly et al., 1996, Neurosurgery 38:523-532) and brains may be harvested after 1 day treatment, sectioned and examined for extent of infarct using TTC staining. Brains may also be examined for the presence of cleaved Caspase-3 immunohistochemically.

Adult C57/B16 mice weighing 23-26 g may be lesioned using the murine stroke model, as described previously for rats. Animals may be placed in a neurological ICU (37° C. incubator) for 90 minutes post reperfusion. They are returned to their cages and examined and sacrificed at indicated time points. All murine stroke experiments may involve blinded assessments of functional and histopathological outcome and may be fully powered (15 animals in each group; 30% reduction in infarct volume results in a 80% power with a p<0.05). Percent ipsilateral infarct may be calculated based on serial scanning of TTC-stained sections and blinded tracing by a trained technician into Adobe Photoshop. Volumes may be calculated using NIH-image. Neurological function may be assessed by a 4 point scale validated in prior studies (Connolly et al., 1996, Neurosurgery 38:523-532). Serial measurements of cerebral blood flow may be made using laser doppler quantification. Values may be recorded immediately after anesthesia, after occlusion of the middle cerebral artery, and immediately after reperfusion. Control groups may include normal animals and sham-operated animals. For protein and RNA extraction, brains may be removed and split into ipsilateral and contralateral hemispheres and flash frozen in liquid nitrogen. For protein analysis brains may be homogenized in RIPA buffer with protease inhibitors. For mRNA analysis, brains may be homogenized in Trizol reagent. Preparation of brain sections may be as described previously.

### 16. Example 11

### Non-Human Primate Ischemia Model

The effect of Pen1-siRNA on cerebral infarction may be examined in non-human primate studies. Cultures of baboon fibroblasts may be used to characterize the efficacy of Pen1-siCasp3. There are no published baboon sequences for Caspase-3 in publicly available databases, but there is high homology among human, macaque and rodent Caspase-3. Pen1-siCasp3 homologous to human, macaque, and rodent may be used in the study. Alternatively, baboon Caspase-3 may be cloned, and siRNA targeting baboon Caspase-3 may be used. Primary cultures of fibroblasts may be treated with Pen1-siCasp3, and Caspase-3 protein expression may be determined with Western blotting. Following the demonstration that Pen1-siCasp3 decreases Caspase-3 mRNA in the cultured baboon fibroblasts, Pen1-siCasp3 may be delivered in vivo in the baboon stroke model. It may then be determined if Pen1-siCasp3 reduces the effect if induced cerebral infarction as compared to baboons not treated with Pen1-Casp3. Baboons may be stroked in the presence or absence of Pen1-siCasp3. 17 animals may constitute each experimental group. Animals may have daily neurologic exams for 30 days, with more detailed exams at 14 and 30 days. Animals may be sacrificed at 30 days and infarct volume may be determined using H&E staining.

Adult male baboons (Papio anubis, 25-35 kg) may be intubated and mechanically ventilated using an inhaled mixture of isoflurane 0.2-0.5% and balanced NO[50%] with O₂[50%], supplemented with an intravenous infusion of fentanyl [50-70 μg/kg/hr], vecuronium, and midazolam. Continuous ICP may be monitored. Core and brain temperature may be maintained with the use of a thermal blanket at ˜37° C. CVP may be maintained with isotonic crystalloid at 5 mm Hg. Adequacy of cerebral ischemia may be confirmed using laser Doppler flowmetry (LDF) (Winfree et al., 2003, Acta Neurochir (Wien) 145:1105-1110). A left transorbital approach may be performed with temporary (75 minutes) clipping of both anterior cerebral arteries proximal to the communicator, as well as, the left internal carotid artery (ICA) at the level of the anterior choroidal artery (Huang et al., 2000, Stroke 31:3054-3063; D'Ambrosio et al., 2004, Methods Enzymol 386:60-73). The recovery period for all animals is 18 hours, during which time the animal may be kept intubated and sedated with propofol under constant surveillance. ICP, CPP, CVP, P_{CO2}, pH, core and brain temperature may be tightly regulated during this time. Sustained ICP above 20 mm Hg may be treated with mannitol (0.5 g/Kg i.v.p.). The primary endpoint is cerebral infarct volume, determined at 72 h by coronal T2 MRI [3 mm thickness with no spacing] (Signa Advantage 1.5 Tesla [General Electric]). In addition, gradient echo, perfusion-weighted (PWI) and MRA images may be obtained. Infarct volume may be determined by planimetric analysis of scanned images (Adobe Photoshop and NIH Image) by two independent blinded observers. Infarct volumes average 30±18% of the ischemic hemisphere. Interobserver variability averages 4.3±0.7% per scan; TTC-staining confirmation varies by only 2.5±0.5%, and delayed scans performed at 10 days in survivors reveals no significant change from early scans (Mack et al., 2003a, Neurol Res 25:846-852). Functional outcomes may be assessed using a validated 100-point task-based scale (Mack et al., 2003b, Neurol Res 25:280-284). The methods described are known in the art, and have been reliably practiced (Mack et al., 2003c, Stroke 34:1994-1999; Mocco et al., 2002, Circ Res 91:907-914).

Intracerebral Clysis in Non-human Primate Stroke Model: On the day of surgery the animals may be taken to the experimental surgery operating rooms, prepped, and anesthetized by the emergency surgery staff. The scalp, neck and upper back may be shaved and prepped under strict sterile conditions. A stereotactic head frame may be attached to the animal's head. A 2-3 cm vertical incision may be made in the frontal scalp 2 cm lateral to midline and the skull exposed. A small 3-5 mm burr hole may be made and the underlying dura mater coagulated. A 26 gauge catheter may be inserted into the caudate nucleus, and the cell-permeable complex may be infused at a rate of 4 μl/min until the entire volume is delivered. After injection, the needle will remain in place for 3 minutes, and may then be retracted over another 3-minute period. After removal of the catheter, the burr hole may be closed with bone wax. Incision sites may be primarily closed with appropriate suture material.

### 17. Example 12

### Use of Pen1-siRNA to Treat Tumors

Delivery of siRNA to the brain may be used to provide a method of studying the function of individual proteins and will also provide a potential therapeutic for diseases of the brain. A rat glioblastoma model (Bruce et al., 2000, Neurosurgery 46:683-691), may be used to examine the effect ICC delivered Pen1-siRNA has on tumor bulk. XIAP has been implicated as a mechanism for promoting tumor growth by blocking cell death (Roa et al., 2003, Clin Invest Med 26:231-242). Knockdown of XIAP may promote death of the tumor cells in a glioblastoma model. Pen1-siRNA targeted to XIAP mRNA, Pen1-siXIAP, has been characterized in vitro (See EXAMPLE-4)

Rat glioblastoma studies: C6 glioma cells may be tested for in vitro sensitivity to knockdown of XIAP. Cells grown at a density of 1 million cells/6 well dish may be treated with 80 nM Pen1-siXIAP and harvested after 1 day treatment for RNA and protein. RNA may be measured by RealTime PCR and protein expression may be determined by Western blotting. The half-life of XIAP in the C6 cells and the sensitivity of these cells to XIAP will be determined, and may be used to define the time parameters of the experiment. Tumors may then be implanted in rats and Pen1-siXIAP may be administered 10 days after tumor implantation. Animal survival may be assessed. Brains may be harvested at the time of death or after 120 days of survival, sectioned and stained with H&E and examined for evidence of tumor.
Tumor Cell Injection: On the day of tumor injection, animals may anesthetized using a rat anesthesia mask to deliver halothane anesthetic as detailed in the rat stroke model methods. A 1.0-1.5 cm incision may be made in the midline of the scalp, and a 1 mm burrhole may be fashioned at a position 1 mm anterior and 3 mm lateral to the bregma. Using a Hamilton microsyringe, 5 microliters of Hanks' balanced salt solution containing 10⁵ tumor cells may be injected into the caudate nucleus (depth of 5 mm) over a period of 60 minutes to prevent reflux along the needle tract. The needle may then be removed over 2 minutes and the skin is closed with three to four interrupted 6-0 vicryl sutures. The animals may be allowed to recover from anesthesia in a temperature controlled incubator maintained at 37° C. for 90 minutes and may be given free access to food and water. Tumor growth occurs over a 10 day period post-operatively, at which point the treatment is administered (see methods for intracerebral clysis in Rats).
Outcome Measures Animal weights may be monitored daily post-operatively, and animals may be sacrificed if a 20% weight loss is observed. Animal survival may be used as the primary end point, and 120 days post-tumor cell implantation is considered long-term survival. All animals undergo brain harvesting at the time of sacrifice. During resection, animals may be reanesthetized, as previously described, and may receive transcardiac perfusion of heparinized saline and 4% formalin. Harvested brains may be incubated in formalin at 4° C. for 72 hours, and then embedded in paraffin. Coronal sections, 4.0 micrometers thick, may be obtained at 20 micrometer intervals. Sections may be stained with hematoxylin and eosin, mounted on glass slides, and examined for gross and microscopic evidence of tumor.

Various publications are cited herein, the contents of which are hereby incorporated by reference in their entireties.

We claim:

1. A method of inhibiting the growth of a tumor in the central nervous system of a subject comprising administering, by convection-enhanced delivery to the central nervous system of the subject, an effective amount of a cell-permeable complex effective in inhibiting expression of a target protein,
wherein the cell-permeable complex comprises (i) a double stranded RNA which is effective in inhibiting the expression of the target protein operably linked to (ii) a cell penetrating peptide; and
wherein the wherein the target protein inhibits apoptosis.

2. The method of claim 1, wherein the double-stranded RNA is a small interfering RNA.

3. The method of claim 1, wherein the double-stranded RNA is selected from the group consisting of small temporal RNA, small nuclear RNA, small nucleolar RNA, short hairpin RNA and microRNA.

4. The method of claim 1, wherein the cell penetrating peptide is selected from the group consisting of penetratin 1, transportan, pIs1, TAT, pVEC, MTS and MAP.

5. The method of claim 1, wherein the target protein is selected from the group consisting of XIAP, cIAP1 and cIAP2.

6. The method of claim 4, wherein the target protein is selected from the group consisting of XIAP, cIAP1 and cIAP2.

7. The method of claim 1, wherein the double stranded RNA is further attached to a label selected from the group consisting of an enzymatic label, a chemical label, and a radioactive label.

8. The method of claim 1, where the convection-based delivery is by clysis.

9. A method of inhibiting cell death associated with ischemia in the central nervous system of a subject comprising administering, by convection-enhanced delivery to the central nervous system of the subject, an effective amount of a cell-permeable complex effective in inhibiting expression of a target protein,
wherein the cell-permeable complex comprises (i) a double stranded RNA which is effective in inhibiting the expression of the target protein operably linked to (ii) a cell penetrating peptide; and
wherein the target protein promotes apoptosis.

10. The method of claim 9, wherein the double-stranded RNA is a small interfering RNA.

11. The method of claim 9, wherein the double-stranded RNA is selected from the group consisting of small temporal RNA, small nuclear RNA, small nucleolar RNA, short hairpin RNA and microRNA.

12. The method of claim 9, wherein the cell penetrating peptide is selected from the group consisting of penetratin 1, transportan, pIs1, TAT, pVEC, MTS and MAP.

13. The method of claim 9, wherein the target protein is selected from the group consisting of caspase 2, caspase 3, caspase 6, caspase 7, caspase 8, caspase 9, PIDD, RAIDD, and NNOS.

14. The method of claim 12, wherein the target protein is selected from the group consisting of caspase 2, caspase 3, caspase 6, caspase 7, caspase 8, caspase 9, PIDD, RAIDD, and NNOS.

15. The composition of claim 9, wherein the double stranded RNA is further attached to a label selected from the group consisting of an enzymatic label, a chemical label, and a radioactive label.

16. The method of claim 9, where the convection-based delivery is by clysis.

17. A composition for instillation into the central nervous system comprising a solution comprising effective amounts of:
(i) a cell-permeable complex;
(ii) albumin and
(iii) a solvent,
wherein the cell-permeable complex comprises a double stranded RNA effective in inhibiting the expression of a target protein encoded by a target mRNA operably linked to a cell penetrating peptide.

18. The composition of claim 17, wherein the double-stranded RNA is a small interfering RNA.

19. The composition of claim 17, wherein the double-stranded RNA is selected from the group consisting of small temporal RNA, small nuclear RNA, small nucleolar RNA, short hairpin RNA and microRNA.

20. The composition of claim 17, wherein the cell penetrating peptide is selected from the group consisting of penetratin 1, transportan, pIs1, TAT, pVEC, MTS and MAP.

21. The composition of claim 17, wherein the target protein is selected from the group consisting of caspase 1, caspase 2, caspase 3, caspase 6, caspase 7, caspase 8, caspase 9, XIAP, cIAP1, cIAP2, PIDD, RAIDD, and NNOS.

22. The composition of claim 20, wherein the target protein is selected from the group consisting of caspase 1, caspase 2, caspase 3, caspase 6, caspase 7, caspase 8, caspase 9, XIAP, cIAP1, cIAP2, PIDD, RAIDD, and NNOS.

23. The composition of claim 17, wherein the double stranded RNA is further attached to a label selected from the group consisting of an enzymatic label, a chemical label, and a radioactive label.

24. A method of inhibiting inflammation in the central nervous system of a subject comprising administering, by convection-enhanced delivery to the central nervous system of the subject, an effective amount of a cell-permeable complex effective in inhibiting expression of a target protein,
wherein the cell-permeable complex comprises (i) a double stranded RNA which is effective in inhibiting the expression of the target protein operably linked to (ii) a cell penetrating peptide; and
wherein the wherein the target protein promotes inflammation.

25. The method of claim 24, wherein the target protein is caspase 1.
